(19) United States
(12) Patent Application Publication
(10) Pub. No.: US 2026/0021090 A1
(43) Pub. Date: Jan. 22, 2026

(54) METHODS OF ADMINISTERING NALBUPHINE

(71) Applicant: Trevi Therapeutics, Inc., New Haven, CT (US)

(72) Inventors: Thomas SCIASCIA, Belmont, MA (US); Shashank ROHATAGI, New Haven, CT (US)

(21) Appl. No.: 18/997,388

(22) PCT Filed: Jul. 24, 2023
(86) PCT No.: PCT/US2023/070849

§ 371 Date: Jan. 21, 2025

Related U.S. Application Data
(60) Provisional application No. 63/369,149, filed on Jul. 22, 2022.

(51) Int. Cl. A61K 31/485; A61P 13/12

(52) U.S. Cl. CPC A61K 31/485; A61P 13/12

(57) ABSTRACT

Provided herein are methods of treating nalbuphine-treatable disorders in an elderly patient.

### CROSS-REFERENCE TO RELATED APPLICATIONS

The present application claims priority to U.S. Provisional Application No. 63/369,149, filed Jul. 22, 2022, the contents of which are hereby incorporated by reference in their entirety for all purposes.

### BACKGROUND

Nalbuphine is a synthetic opioid that acts as a competitive μ-opioid receptor antagonist while producing agonist effects at the κ-opioid receptor. Nalbuphine is currently available as an injectable generic medication approved for the treatment of moderate to severe pain, a supplement to balanced anesthesia, for pre-operative and post-operative analgesia and obstetrical analgesia during labor and delivery.

Nalbuphine can be useful in treating a variety of other conditions, including chronic pruritic conditions (such as, prurigo nodularis, uremic pruritus, and chronic liver disease) and neurologically mediated conditions (such as chronic cough and levodopa-induced dyskinesia), all of which conditions that are highly prevalent in the elderly population. However, due to age-related changes in pharmacokinetics, elderly patients may have increased risk of complications or toxicities with nalbuphine use. To date, there are no clear guidelines to assist a physician in determining an effective treatment for elderly patients suffering from the nalbuphine-treatable disorders.

Thus, there is a need for safe and effective methods for treating elderly patients with nalbuphine.

### SUMMARY OF THE DISCLOSURE

The present disclosure provides methods of treating a nalbuphine-treatable disorder in an elderly patient by administering a therapeutically effective daily dose of nalbuphine, or a pharmaceutically acceptable salt or ester thereof. In embodiments, the elderly patient is administered a daily dose that is about 40% to about 60% of the daily dose administered to a non-elderly patient treated for the same nalbuphine-treatable disorder. In embodiments, the administered daily dose is about 50% of the daily dose administered to a non-elderly patient treated for the same nalbuphine-treatable disorder.

In embodiments, the nalbuphine-treatable disorder is selected from the group consisting of idiopathic pulmonary fibrosis (IPF), chronic cough, uremic pruritus, prurigo nodularis, and levodopa-induced dyskinesia (LID).

In embodiments, the elderly patient is at least 65 years old. In embodiments, the elderly patient is at least 75 years old. In embodiments, the non-elderly patient is 18 to 74 years old. In embodiments, the non-elderly patient is 18 to 64 years old.

In embodiments, the elderly patient is administered a daily dose of about 28 mg to about 170 mg of an Equivalent Amount of Nalbuphine Free Base.

In embodiments, the nalbuphine is administered once daily. In embodiments, the nalbuphine is administered twice daily. In embodiments, the method further comprises titrating the dose for about 7 to 30 days. In embodiments, the method further comprises titrating the dose for about 7 to 14 days.

In embodiments, the titration comprises administration of an Equivalent Amount of Nalbuphine Free Base according to the following schedule:

| Day | AM dosage (mg) | PM dosage (mg) |
|---|---|---|
| Day 1 | 0 | about 14 |
| Day 2 | 0 | about 14 |
| Day 3 | about 14 | about 14 |
| Day 4 | about 14 | about 14 |
| Day 5 | about 14 | about 27 |
| Day 6 | about 27 | about 27 |
| Day 7 | about 27 | about 27 |
| Day 8 | about 27 | about 40 |
| Day 9 | about 40 | about 40 |
| Day 10 | about 40 | about 40 |
| Day 11 | about 40 | about 54 |
| Day 12 | about 54 | about 54 |
| Day 13 | about 54 | about 54 |
| Day 14 | about 54 | about 81. |

In embodiments, the present disclosure provides methods of treating a nalbuphine-treatable disorder in a patient in need thereof, wherein the methods comprise the steps of: (a) determining the patient's age; (b) administering a daily dose 28 mg to about 170 mg of an Equivalent Amount of Nalbuphine Free Base to an elderly patient, and (c) administering a daily dose 54 mg to about 324 mg of an Equivalent Amount of Nalbuphine Free Base to a non-elderly patient, wherein nalbuphine treatable conditions is selected from the group consisting of IPF cough, uremic pruritus, prurigo nodularis, and levodopa-induced dyskinesia (LID).

In embodiments, the daily dose administered to an elderly patient is about 40% to about 60% of the daily dose administered to a non-elderly patient treated for the same nalbuphine-treatable disorder. In embodiments, the daily dose administered to an elderly patient is about 50% of the daily dose administered to a non-elderly patient treated for the same nalbuphine-treatable disorder.

In embodiments, the elderly patient is at least 65 years old. In embodiments, the elderly patient is at least 75 years old. In embodiments, the non-elderly patient is 18 to 64 years old. In embodiments, the non-elderly patient is 18 to 74 years old.

In embodiments, the elderly patient is administered a daily dose of about 28 mg to about 170 mg of an Equivalent Amount of Nalbuphine Free Base.

In embodiments, the nalbuphine is administered twice daily. In embodiments, the nalbuphine is administered once daily. In embodiments, the method further comprises titrating the dose for about 7 to 30 days. In embodiments, the method further comprises titrating the dose for about 7 to 14 days.

In embodiments, the titration comprises administration of an Equivalent Amount of Nalbuphine Free Base according to the following schedule

| Day | AM dosage (mg) | PM dosage (mg) |
|---|---|---|
| Day 1 | 0 | about 14 |
| Day 2 | 0 | about 14 |
| Day 3 | about 14 | about 14 |
| Day 4 | about 14 | about 14 |
| Day 5 | about 14 | about 27 |
| Day 6 | about 27 | about 27 |
| Day 7 | about 27 | about 27 |
| Day 8 | about 27 | about 40 |
| Day 9 | about 40 | about 40 |
| Day 10 | about 40 | about 40 |
| Day 11 | about 40 | about 54 |
| Day 12 | about 54 | about 54 |
| Day 13 | about 54 | about 54 |
| Day 14 | about 54 | about 81. |

### Definitions

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, example and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “adverse event” (AE) as used herein is defined as any untoward medical occurrence in a clinical investigation patient reported on or after the first screening date. An AE does not necessarily have to have a causal relationship with the treatment. An AE can therefore be any unfavorable and unintended sign (including an abnormal laboratory finding), symptom whether or not related to the medicinal (investigational) product, or disease temporally associated with the use of a medicinal (investigational) product. Typical adverse events include nausea, vomiting, somnolence, and dizziness. In accordance with the present disclosure, the rate of adverse events after the treatment is substantially the same as the rate of adverse events after administering a placebo for the same period of time.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, in some embodiments, an effective amount of nalbuphine, or a pharmaceutically acceptable salt or ester thereof, is that amount which is required to reduce at least one symptom of pruritus in a patient, e.g., the amount required to reduce the itching sensation in a patient. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” as used herein with regard to a patient, refers to improving at least one symptom of the patient's disorder. Treating can be improving, or at least partially ameliorating a disorder.

The term “therapeutic effect” as used herein refers to a desired or beneficial effect provided by the method and/or the composition. For example, the method for treating pruritus provides a therapeutic effect when the method reduces at least one symptom of pruritus, e.g., itching sensation, in a patient.

### DETAILED DESCRIPTION

Prurigo Nodularis (PN) is an intensely pruritic dermatologic condition characterized by the presence of itchy nodules which often have associated excoriations and ulcerations, together with other types of pruriginous lesions such as papules and/or plaques. These lesions can involve extensive areas of the trunk, arms or legs. The pathophysiology of PN is considered by experts to be due to a self-re-enforcing itch-scratch cycle that persists and worsen due to central neuronal sensitization. Not wishing to be bound by theory, it is believed that the sensation of itch and the urge to scratch are mediated in part by κ- and μ-opioid receptor expression in the reward centers of the brain as well as the spinal cord, skin and peripheral nervous system. Therefore, therapeutic intervention through modulation of the opioid receptors could potentially break the itch-scratch cycle by interrupting the positive neuronal feedback loop as well as the interactions between the nervous and immune systems.

Nalbuphine is a synthetic opioid that acts as a competitive antagonist of the μ-opioid receptor while producing agonist effects at the κ-opioid receptor. Other nalbuphine-treatable disorders include, but are not limited to, IPF cough, uremic pruritus, and levodopa-induced dyskinesia (LID), all of which are highly prevalent in the elderly population.

Generally, elderly patients report age-related changes or reductions in pharmacokinetics such as altered drug absorption, distribution, metabolism and/or excretion rates. The alterations in metabolic activities by aging can significantly impact the efficacy and safety of drugs. There is little information in the literature relating to nalbuphine dose ranging in elderly patients, representing a significant gap in the medical literature given that the extent of PK alteration cannot be predicted.

The present disclosure provides, among other things, methods of treating a nalbuphine-treatable disorder in an elderly patient in need thereof. In embodiments, the methods comprise administering a therapeutically effective daily dose of nalbuphine to an elderly patient.

Throughout the present disclosure, the methods and doses used herein are generally expressed in terms of an Equivalent Amount of Nalbuphine Free Base for the “treatment of nalbuphine-treatable disorders” without specifying a particular nalbuphine-treatable disorder. For clarity, the present disclosure contemplates embodiments where methods and doses disclosed herein are effective for the treatment of any particular nalbuphine-treatable disorder, for example, chronic cough, pruritus, prurigo nodularis, uremic pruritus, pruritus associated with liver disease, tardive dyskinesia, Huntington's disease, and levodopa-induced dyskinesia (LID).

In embodiments, the present disclosure provides methods of treating pruritus in elderly patients. In embodiments, the present disclosure provides methods of treating prurigo nodularis in elderly patients. In embodiments, the present disclosure provides methods of treating uremic pruritus in elderly patients. In embodiments, the present disclosure provides methods of treating cough, breathlessness or dyspnea in elderly patients. In embodiments, the present disclosure provides methods of treating idiopathic pulmonary fibrosis (IPF) cough, breathlessness or dyspnea in elderly patients. In embodiments, nalbuphine HCl is administered in the methods of the present disclosure. The present disclosure also contemplates the treatment of the nalbuphine-treatable disorders using the nalbuphine doses described in U.S. Pat. No. 8,637,538, and U.S. Patent Publication Nos. 2014/0179727; 2018/0008592; 2018/0125840; and 2020/0022974, which are incorporated by reference herein in their entireties. In embodiments, the cough, breathlessness, or dyspnea associated with a particular condition such as IPF, refractory chronic cough, unexplained chronic cough, hypersensitivity pneumonitis, sarcoidosis, asbestosis, bronchiolitis obliterans, histiocytosis X, chronic eosinophilic pneumonia, collagen vascular disease, granulomatous vasculitis, Goodpasture's syndrome and, pulmonary alveolar proteinosis, COPD (such as COPD is associated with a condition such as emphysema, chronic bronchitis and Alpha-1-antitrypsin (AAt) deficiency or COPD associated with an irritant such as cigarette smoke, secondhand smoke, pipe smoke, air pollution and workplace exposure to dust, smoke or fumes) and other conditions described herein. In embodiments, the chronic cough is selected from refractory chronic cough, unexplained chronic cough, unexplained and refractory chronic cough, idiopathic chronic cough, cough hypersensitivity syndrome, hypertussia, allotussia and neurogenic cough as well as suppression of the sensation of the urge to cough. In embodiments, methods of the present disclosure are used for the treatment of cough, breathlessness, or dyspnea associated with IPF, uremic pruritus, prurigo nodularis, and/or levodopa-induced dyskinesia (LID).

In embodiments, the elderly patient is 55 years of age, 65 years of age, 75 years of age, or older. In embodiments, the elderly patient is at least about 55 years old, at least about 56 years, at least about 57 years old, at least about 58 years old, at least about 59 years old, at least about 60 years old, at least about 61 years old, at least about 62 years old, at least about 63 years old, at least about 64 years old, at least about 65 years old, at least about 66 years old, at least about 67 years old, at least about 68 years old, at least about 69 years old, at least about 70 years old, at least about 71 years old, at least about 72 years old, at least about 73 years old, at least about 74 years old, at least about 75 years old, at least about 76 years old, at least about 77 years old, at least about 78 years old, at least about 79 years old, at least about 80 years old, at least about 81 years old, at least about 82 years old, at least about 83 years old, at least about 84 years old, at least about 85 years old, at least about 86 years old, at least about 87 years old, at least about 88 years old, at least about 89 years old, at least about 90 years old, at least about 91 years old, at least about 92 years old, at least about 93 years old, at least about 94 years old, at least about 95 years old, at least about 96 years old, at least about 97 years old, at least about 98 years old, at least about 99 years old, or at least about 100 years old, including any values and sub-ranges therebetween. In embodiments, the elderly patient is at least about 75 years old. In embodiments, the elderly patient is at least about 70 years old. In embodiments, the elderly patient is at least about 65 years old.

In embodiments, the elderly patient is administered a lower daily dose than the daily dose administered to a non-elderly patient treated for the same nalbuphine-treatable disorder. In embodiments, the non-elderly patient is 74 years old or younger. In embodiments, the non-elderly patient is about 0 to 74 years old, 2 to 74 years old, 4 to 74 years old, 6 to 74 years old, 8 to 74 years old, 10 to 74 years old, 12 to 74 years old, 14 to 74 years old, 16 to 74 years old, 18 to 74 years old, 20 to 74 years old, 22 to 74 years old, 24 to 74 years old, 0 to 72 years old, 2 to 72 years old, 4 to 72 years old, 6 to 72 years old, 8 to 72 years old, 10 to 72 years old, 12 to 72 years old, 14 to 72 years old, 16 to 72 years old, 18 to 72 years old, 20 to 72 years old, 22 to 72 years old, 24 to 72 years old, 18 to 69 years old, 18 to 68 years old, 18 to 66 years old, 0 to 64 years old, 2 to 64 years old, 4 to 64 years old, 6 to 64 years old, 8 to 64 years old, 10 to 64 years old, 12 to 64 years old, 14 to 64 years old, 16 to 64 years old, 18 to 64 years old, 20 to 64 years old, 22 to 64 years old, 23 to 64 years old, or 24 to 64 years old, including any sub-ranges and values therebetween. In embodiments, the non-elderly patient is about 18 to 74 years old. In embodiments, the non-elderly patient is about 18 to 72 years old. In embodiments, the non-elderly patient is about 18 to 69 years old. In embodiments, the non-elderly patient is about 18 to 64 years old.

In embodiments, the elderly patient is administered a daily dose of about 40% to about 60% of the daily dose administered to a non-elderly patient treated for the same nalbuphine-treatable disorder. In embodiments, the elderly patient is administered a daily dose of about 30% to about 70%, about 40% to about 60%, about 45% to about 55%, or about 50% to about 55% of the daily dose administered to a non-elderly patient treated for the same nalbuphine-treatable disorder, including any sub-ranges and values therebetween. In embodiments, the elderly patient is administered a daily dose of about 20% to about 60%, about 22% to about 60%, about 24% to about 60%, about 26% to about 60%, about 28% to about 60%, about 30% to about 60%, about 32% to about 60%, about 34% to about 60%, about 36% to about 60%, about 38% to about 60%, about 40% to about 60%, about 42% to about 60%, about 44% to about 60% or about 46% to about 60%, about 48% to about 60%, or about 50% to about 60% of the daily dose administered to a non-elderly patient treated for the same nalbuphine-treatable disorder, including any sub-ranges and values therebetween. In embodiments, the elderly patient is administered a daily dose of about 40% to about 60% of the daily dose administered to a non-elderly patient treated for the same nalbuphine-treatable disorder. In embodiments, the elderly patient is administered a daily dose of about 50% of the daily dose administered to a non-elderly patient treated for the same nalbuphine-treatable disorder. In embodiments, the elderly patient is administered a daily dose of about 53% of the daily dose administered to a non-elderly patient treated for the same nalbuphine-treatable disorder. In embodiments, the elderly patient is administered a daily dose of about 60%, about 59%, about 58%, about 57%, about 56%, about 55%, about 54%, about 53%, about 52%, about 51%, about 50%, about 49%, about 48%, about 47%, about 46 about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27, about 26%, about 25, about 24%, about 23%, about 22%, about 21%, or about 20% of the daily dose administered to a non-elderly patient treated for the same nalbuphine-treatable disorder, including any values and sub-ranges therebetween.

In embodiments, the elderly patient is administered a daily dose of about 40% to about 60%, or about 50% of the daily dose administered to a non-elderly patient treated for the same nalbuphine-treatable disorder. For example, in embodiments, an elderly patient is administered a daily dose of about 27 mg to about 162 mg of nalbuphine while a non-elderly patient is administered a daily dose of about 54 mg to about 324 mg of nalbuphine for treating the same nalbuphine-treatable disorder.

In embodiments, the present disclosure provides methods for administering a lower dose of nalbuphine (e.g., about 40% to about 60% lower, or about 50% lower) to an elderly patient in need of treatment for a nalbuphine-treatable disorder, compared to an otherwise physiologically identical non-elderly patient.

In embodiments, the present disclosure also provides a method of treating a nalbuphine-treatable disorder in a patient in need thereof, wherein the method comprises the steps of: (a) determining the patient's age; (b) administering a daily dose 28 mg to about 170 mg of an Equivalent Amount of Nalbuphine Free Base to an elderly patient; and (c) administering a daily dose 54 mg to about 324 mg of an Equivalent Amount of Nalbuphine Free Base to a non-elderly patient, wherein nalbuphine treatable conditions is selected from the group consisting of IPF cough, uremic pruritus, prurigo nodularis, and levodopa-induced dyskinesia (LID).

In accordance with embodiments of the present disclosure, the method provides a therapeutic effect without producing a substantial adverse event. In embodiments, the rate of adverse events after the treatment with nalbuphine is substantially the same as the rate of adverse events after administering a placebo for the same period of time. In accordance with embodiments of the present disclosure, the method of treating pruritus does not produce a substantial aquaretic effect.

### Nalbuphine

Nalbuphine as employed in the present methods can form a part of a pharmaceutical composition by combining nalbuphine, or a pharmaceutically acceptable salt, solvate or ester thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended-release formulation.

Nalbuphine HCl (17-(cyclobutylmethyl)-4,5α-epoxymorphinian-3, 6α, 14-triol, hydrochloride) is a synthetic opioid. Structurally, nalbuphine is a derivative of 14 hydroxymorphine.

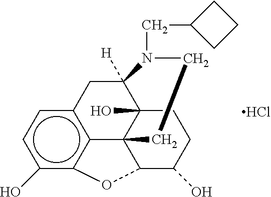

Nalbuphine HCl is currently available only as a generic medication in an injectable form. An injectable form of nalbuphine has been available as an approved drug formulation since 1978. Nubain® was the innovator brand injectable form of nalbuphine on which the presently sold generic bioequivalent injectable formulations are based. The injectable formulation is currently approved for use in the relief of moderate to severe pain, a supplement to balanced anesthesia, for pre-operative and post-operative analgesia and obstetrical analgesia during labor and delivery.

The present disclosure also provides pharmaceutically acceptable esters of nalbuphine. The term “ester” denotes a derivative of the agent containing an ester functional group (as described herein), which is capable of releasing the agent when the ester form is administered to a patient. Release of the active ingredient occurs in vivo. Pharmaceutically acceptable esters can be prepared by techniques known to one skilled in the art. These techniques generally modify appropriate functional groups in a given compound. These modified functional groups however regenerate original functional groups by metabolism of the compound in vivo. Esters include compounds wherein a hydroxy, carboxylic, or a similar group is modified.

Suitable pharmaceutically acceptable esters for a hydroxyl group include inorganic esters such as phosphate esters and α-acyloxyalkyl ethers and related compounds which, as a result of in vivo hydrolysis of the ester, provide the parent hydroxy group. In vivo hydrolyzable ester forming groups for hydroxy include alkanoyl (e.g., C₁₋₁₀ linear, branched or cyclic alkyl), benzoyl, phenylacetyl and substituted benzoyl and phenylacetyl, alkoxycarbonyl (to give alkyl carbonate esters), dialkylcarbamoyl and N—(N, N-dialkylaminoethyl)-N-alkylcarbamoyl (to give carbamates), N, N-dialkylaminoacetyl and carboxyacetyl.

In embodiments, the nalbuphine used in the formulations and methods of the present disclosure is a pharmaceutically acceptable co-crystal of nalbuphine.

### Formulations

The methods of the present disclosure can employ various formulations for administration to patients, e.g., humans and animals in unit dosage forms, such as tablets, capsules, pills, powders, granules, sterile parenteral solutions or suspensions, and oral solutions or suspensions, and oil-water emulsions containing suitable quantities of nalbuphine, or pharmaceutically acceptable salts or esters thereof.

Oral pharmaceutical dosage forms can be either solid or liquid. The solid dosage forms can be tablets, capsules, granules, and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In embodiments, the oral dosage form may be an osmotic-controlled release oral delivery system (OROS). In embodiments, the oral dosage form may include matrix-embedded dosage forms or related devices. In embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Emulsions can be either oil-in water or water-in-oil. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, can include organic acids and a source of carbon dioxide. Coloring and flavoring agents can be used in all of the above dosage forms.

Parenteral administration of the formulations of the present disclosure comprise intravenous, subcutaneous and intramuscular administrations of immediate, sustained (e.g., depot), extended, and/or modified release formulations (e.g., as described herein). Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the pharmaceutically active compound can be adjusted so that an injection provides an effective amount to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing nalbuphine, or a pharmaceutically acceptable salt or ester thereof, is an effective mode of administration.

Pharmaceutical dosage forms for rectal administration can be rectal suppositories, capsules and tablets for systemic effect. Rectal suppositories as used herein mean solid bodies for insertion into the rectum which melt or soften at body temperature releasing the pharmacologically and/or therapeutically active ingredients contained in the composition of this disclosure. Pharmaceutically acceptable substances utilized in rectal suppositories are bases or vehicles and agents to raise the melting point. Examples of bases include cocoa butter (theobroma oil), glycerin-gelatin, carbowax, polyoxyethylene glycol and mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 mg. Tablets and capsules for rectal administration can be manufactured using the same pharmaceutically acceptable substance and by the same methods as for formulations for oral administration.

The compositions can be suspended in micronized or other suitable form or can be derivatized to produce a more soluble active product. The form of the resulting composition depends upon a number of factors, including the intended mode of administration and the solubility of the nalbuphine, or a pharmaceutically acceptable salt or ester thereof, in the selected carrier or vehicle. The effective concentration is sufficient for treating or alleviating nalbuphine-treatable disorders (e.g., pruritus) and can be empirically determined. The concentration is generally greater than the concentration for systemic administration of the compound.

The resulting mixture can be a solution, suspension, emulsion or the like, and can be formulated as a cream, gel, ointment, emulsion, solution, elixir, lotion, suspension, tincture, paste, foam, aerosol, irrigation, spray, suppository, bandage, or any other formulation suitable for topical administration. Modes of administration can include topical application to the skin, scalp, eyes, and/or nasal, buccal or sublingual mucosa.

Pharmaceutical and cosmetic carriers or vehicles suitable for administration of the compositions include any such carriers known to those skilled in the art to be suitable for the particular mode of administration. The nalbuphine, or a pharmaceutically acceptable salt or ester thereof, can be included in the carriers in amounts sufficient to exert a therapeutically useful effect without serious toxic effects on the treated individual.

To formulate these compositions, a weight fraction of nalbuphine, or a pharmaceutically acceptable salt or ester thereof, is dissolved, suspended, dispersed or otherwise mixed in a selected vehicle at an effective concentration such that the nalbuphine-treatable disorder condition is relieved or ameliorated. Generally, emollient or lubricating vehicles that help hydrate the skin are more preferred than volatile vehicles, such as ethanol, that dry the skin. Examples of suitable. In embodiments, the composition comprises bases or vehicles for preparing compositions for use with human skin, wherein the base or vehicle is petrolatum, petrolatum plus volatile silicones, lanolin, cold cream (USP), or hydrophilic ointment (USP), or a mixture thereof.

The compositions employed in the present methods can relieve nalbuphine-treatable disorders when applied to the skin. Relief can be temporary or permanent, and can even be evident after a single dose of the composition. When the composition is administered in a form other than a topical preparation, it should be administered in an amount sufficient to provide relief from nalbuphine-treatable disorders that is within safety guidelines established by the FDA. Determining the appropriate amount to administer to a patient is within the skill of the person of ordinary skill in the art in association with teachings provided by the present disclosure.

In embodiments, the compositions are solutions for topical administration and comprise an amount of the composition effective to deliver a tolerable and therapeutically effective amount, typically at a concentration of between about 0.01% w/w to about 5% w/w. The balance of the solution is water, a suitable organic solvent or other suitable solvent or buffer. In embodiments, the compositions that are formulated as solutions or suspensions are applied to the skin, or are formulated as an aerosol or foam and applied to the skin as a spray-on. In embodiments, the aerosol compositions comprise about 25% to 80% w/w or about 30% to 50% w/w, of a suitable propellant. In embodiments, gel compositions are formulated by admixing a suitable thickening agent to the solution or suspension.

In embodiments, the compositions are in solid forms for topical application and are formulated as stick-type compositions intended for application to the lips or other parts of the body. Such compositions comprise an effective amount of nalbuphine, or a pharmaceutically acceptable salt, solvate or ester thereof. In embodiments, the amount of the nalbuphine, or a pharmaceutically acceptable salt or ester thereof, is about 0.01% w/w to about 5% w/w. In embodiments, the solids comprise about 40% to 98% w/w, or about 50% to 90% w/w, of emollients. In embodiments, the compositions further comprise about 1% to 20% w/w, or about 5% to 15% w/w of a suitable thickening agent, and/or emulsifiers and water or buffers.

In embodiments, the nalbuphine, or pharmaceutically acceptable salt, solvate or ester thereof, is present in the composition in an amount of about 1 mg to 200 mg, including in an amount of from about 1 mg, about 2 mg, about 3 mg, about 4 mg, about 5 mg, about 6 mg, about 7 mg, bout 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 60 mg, about 65 mg, about 70 mg, about 75 mg, about 80 mg, about 85 mg, about 90 mg, about 95 mg, about 100 mg, about 105 mg, about 108 mg, about 110 mg, about 120 mg, about 130 mg, about 135 mg, about 140 mg, about 145 mg, about 150 mg, about 155 mg, about 160 mg, about 165 mg, or about 170 mg, including all sub-ranges and values therebetween.

In embodiments, the nalbuphine, or a pharmaceutically acceptable salt thereof, is present in the composition in an amount of about 2 mg to about 170 mg; about 7 mg to about 170 mg; about 14 mg to about 160 mg; about 28 mg to about 150 mg; about 40 mg to about 140 mg; about 50 mg to about 130 mg; about 60 mg to about 120 mg; about 70 mg to about 110 mg, or about 80 mg to about 100 mg, including all sub-ranges and values therebetween. In embodiments, the nalbuphine, or pharmaceutically acceptable salt, solvate or ester thereof, is present in the composition in an amount of about 1 mg to about 80 mg, about 5 mg to about 75 mg; about 10 mg to about 70 mg; about 20 mg to about 60 mg; about 40 mg to about 60 mg; or about 45 mg to about 55 mg, including all sub-ranges and values therebetween. In embodiments, the pharmaceutically acceptable salt of nalbuphine, e.g., nalbuphine HCl, is present in the composition in an amount of about 4 mg, 8 mg, 15 mg, about 30 mg, about 60 mg, about 90 mg, about 100 mg, about 120 mg, about 150 mg, or about 170 mg, including all sub-ranges and values therebetween.

In embodiments, the compositions comprising a pharmaceutically acceptable salt of nalbuphine express the amount of nalbuphine in said compositions as the Equivalent Amount of Nalbuphine Free Base, which is the calculated amount of nalbuphine free base in the composition based on the actual amount of the pharmaceutically acceptable salt of nalbuphine in the composition. The amount of the Equivalent Amount of Nalbuphine Free Base in a composition will vary within the manufacturing process, and the compositions of the present disclosure encompass pharmaceutically-acceptable deviations (i.e., FDA-acceptable) from the nalbuphine content that is recited in the present disclosure.

In embodiments, nalbuphine is in the form of any pharmaceutically acceptable salt known in the art. Exemplary pharmaceutically acceptable salts include without limitation hydrochloric, sulfuric, nitric, phosphoric, hydrobromic, maleic, malic, ascorbic, citric, tartaric, pamoic, lauric, stearic, palmitic, oleic, myristic, lauryl sulfuric, napthalenesulfonic, linoleic, linolenic acid, and the like. Some embodiments include the hydrochloride salt of nalbuphine.

The following table shows the Equivalent Amount of Nalbuphine Free Base for compositions containing 15 mg, 30 mg, 60 mg, 90 mg, 120 mg, 170 mg and 190 mg of nalbuphine HCl:

| | |
|---|---|
| Amount of | Equivalent Amount of |
| nalbuphine HCl | Nalbuphine Free Base |

| | | |
|---|---|---|
| 4 | mg | 3.6¹ |
| 8 | mg | 7.2 |
| 15 | mg | 13.6 |
| 30 | mg | 27.2 |
| 60 | mg | 54.4 |
| 90 | mg | 81.6 |
| 120 | mg | 108.8 |
| 170 | mg | 154.1 |
| 190 | mg | 172.3 |

¹The amount of Equivalent Amount of Nalbuphine Free Base is rounded to the nearest 0.1 decimal place using the equation below.

Throughout the present disclosure, the amount of nalbuphine in a composition is generally expressed in terms of Equivalent Amount of Nalbuphine Free Base. However, the present disclosure contemplates embodiments where the nalbuphine is present in a pharmaceutically acceptable salt and/or ester and provides the amount of nalbuphine hydrochloride present in a composition. The about the same Equivalent Amount of Nalbuphine Free Base as the embodiments that are expressly described herein. For example, about 251 mg of nalbuphine citrate (FW=549.57 g/mol) provides about the same Equivalent Amount of Nalbuphine Free Base as about 180 mg of nalbuphine hydrochloride. The Equivalent Amount of Nalbuphine Free Base in said compositions may be calculated by the following formula:

Equivalent Amount of Nalbuphine Free Base=

$$\frac{\begin{matrix}Mass of Pharmaceutically Acceptable Salt ( g ) \times \\ 357.45 ( Formula Weight of Nalbuphine Free Base , \frac{g}{mol} )\end{matrix}}{Formula Weight of Pharmaceutically Acceptable Salt ( \frac{g}{mol} )}$$

The Equivalent Amount of Nalbuphine Free Base content of the dosage form calculated using the equation above may be adjusted by a pharmaceutically acceptable amount (for example, within an amount permitted by FDA safety standards, which, in embodiments, is 1% or less of the calculated Equivalent Amount of Nalbuphine Free Base) to allow product labeling using a whole number integer when referencing the dosage strength. For example, in embodiments, the calculated Equivalent Amount of Nalbuphine Free Base for 190 mg of nalbuphine hydrochloride is 172.3 mg, but adjusted to 170 mg. In embodiments, the nalbuphine content of the composition is adjusted for a product labelling of Equivalent Amount of Nalbuphine Free Base.

### Sustained Release

In embodiments, the nalbuphine formulations employed in the present methods are oral sustained release nalbuphine formulations as described in U.S. Patent Publication Nos. 2019/0117576, 2019/0099416, 2015/0359789 2009/0030026, and 2007/0048376; and PCT Publication Nos. 2015/192071 and 2007/025005; each of which is incorporated herein by reference in their entireties.

“Sustained release” or “extended release” means that the nalbuphine, or pharmaceutically acceptable salt, solvate or ester thereof, is released from the formulation at a controlled rate so that therapeutically beneficial blood levels (but below toxic levels) of the nalbuphine, or pharmaceutically acceptable salt, solvate or ester thereof, are maintained over an extended period of time. Alternatively, “sustained release” or “extended release” means that the desired pharmacologic effect is maintained over an extended period of time.

The half-life of nalbuphine injectable formulations (i.e., IV or IM or SC) has been reported to be relatively short, only about 2-3 hours. In embodiments, the present methods employ oral sustained release formulations of nalbuphine, which comprise an effective amount of nalbuphine, or a pharmaceutically acceptable salt, solvate or ester thereof. In embodiments, the oral sustained release formulations provide a controlled release and a lower Cₘₐₓ of the nalbuphine, or a pharmaceutically acceptable salt thereof, over a longer period than observed for bolus injections or immediate release oral formulations (e.g., at least about 8-12 hours). Reducing the frequency of dosing provides the potential for enhanced patient convenience and compliance with the present methods. The lower dosing frequency also has the potential to provide reduced side effects because the patient may be exposed to lower peak concentrations of agent over time. In embodiments, the methods of the present disclosure provide an anti-pruritic effect as well as breaking the cycle effect, e.g., the itchy sensation does not return after certain treatment period.

In embodiments, the nalbuphine compositions used in the methods of the present disclosure include nalbuphine, or a pharmaceutically acceptable salt, solvate or ester thereof, and a sustained release delivery system. The sustained release delivery system comprises (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. In embodiments, the compositions include nalbuphine, or a pharmaceutically acceptable salt, solvate or ester thereof, and a sustained release delivery system including a hydrophobic compound in a sustained release system. In embodiments, the nalbuphine can be homogeneously dispersed in the sustained release delivery system.

In embodiments, the ratio of nalbuphine or pharmaceutically acceptable salt, solvate or ester thereof in the compositions to the sustained release delivery system is about 4:1 to about 1:25. In embodiments, the ratio of nalbuphine or pharmaceutically acceptable salt, solvate or ester thereof to the sustained release delivery system is about 2.5:1 to about 1:4. In embodiments, the ratio of nalbuphine or pharmaceutically acceptable salt, solvate or ester thereof to the sustained release delivery system is about 5:1 to about 1:5, about 4:1 to about 1:4, about 3:1 to about 1:3, about 2:1 to about 1:2, about 1:1 to about 1:5, about 1:1 to about 1:4, about 1:1 to about 1:3, about 1:1 to about 1.2, or about 1:2 to about 1:3, including any values or ranges therebetween. In embodiments, the ratio of nalbuphine or pharmaceutically acceptable salt, solvate or ester thereof to the sustained release delivery system is about 1:1, about 1:2, about 1:2.5, about 1:3, about 1:4, or about 1:5, including any values or ranges therebetween.

In embodiments, at least one hydrophilic compound is present in the sustained release delivery system in an amount of about 5% to about 80% by weight; the at least one cross-linking agent is present in the sustained release delivery system in an amount of about 0.5% to about 80% by weight; and the at least one pharmaceutical diluent is present in the sustained release delivery system in an amount of about 20% to about 80% by weight. In embodiments, the at least one hydrophilic compound is present in the sustained release delivery system in an amount of about 8% to about 31% by weight; the at least one cross-linking agent is present in the sustained release delivery system in an amount of about 12% to about 47% by weight; and the at least one pharmaceutical diluent is present in the sustained release delivery system in an amount of about 20% to about 78% by weight. In embodiments, the at least one hydrophilic compound is present in the sustained release delivery system in an amount of about 10% to about 20% by weight; the at least one cross-linking agent is present in the sustained release delivery system in an amount of about 15% to about 25% by weight; and the at least one pharmaceutical diluent is present in the sustained release delivery system in an amount of about 50% to about 85% by weight. In embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, or about 36% by weight, including any values or ranges therebetween; the at least one cross-linking agent is present in the sustained release delivery system in an amount of about 10%, about 11%, about 12%, about 13%, about 14%, about 15%, about 16%, about 17%, about 18%, about 19%, about 20%, about 22%, about 24%, about 26%, about 28%, about 30%, about 32%, about 33%, about 34%, or about 35% by weight, including any values or ranges therebetween; and the at least one pharmaceutical diluent is present in the sustained release delivery system in an amount of about 40%, about 45%, about 50%, about 55%, about 60%, about 65%, about 70%, about 80%, or about 85% by weight, including any values or ranges therebetween.

In embodiments, the at least one hydrophilic compound is present in the sustained release delivery system in an amount of about 10%, about 11%, about 12%, about 13%, about 14%, about 15%, about 16%, about 17%, about 18%, about 19%, or about 20%, including any values or ranges therebetween, by weight; the at least one cross-linking agent is present in the sustained release delivery system in an amount of about 15%, about 16%, about 17%, about 18%, about 19%, about 20%, or about 22% by weight, including any values or ranges therebetween; and the at least one pharmaceutical diluent is present in the sustained release delivery system in an amount of about 55%, about 60%, about 65%, about 70%, about 80%, or about 85% by weight, including any values or ranges therebetween. In embodiments, the at least one hydrophilic compound is present in the sustained release delivery system in an amount of about 8%, about 12%, or about 20% by weight; the at least one cross-linking agent is present in the sustained release delivery system in an amount of about 12%, about 18%, or about 30% by weight; and the at least one pharmaceutical diluent is present in the sustained release delivery system in an amount of about 40%, about 60%, or about 70% by weight.

In embodiments, nalbuphine is in the form of any pharmaceutically acceptable salt known in the art. In embodiments, nalbuphine is in the form of hydrochloric, sulfuric, nitric, phosphoric, hydrobromic, maleic, malic, ascorbic, citric, tartaric, pamoic, lauric, stearic, palmitic, oleic, myristic, lauryl sulfuric, napthalenesulfonic, linoleic, or linolenic acid. In embodiments, nalbuphine is in the form of a hydrochloride salt.

The sustained release delivery system comprises at least one hydrophilic compound. The hydrophilic compound preferably forms a gel matrix that releases the nalbuphine or the pharmaceutically acceptable salt, solvate or ester thereof at a sustained rate upon exposure to liquids. The rate of release of the nalbuphine or the pharmaceutically acceptable salt, solvate or ester thereof from the gel matrix depends on the drug's partition coefficient between the components of the gel matrix and the aqueous phase within the gastrointestinal tract. In embodiments, the weight ratio of nalbuphine to hydrophilic compound is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including any values or ranges therebetween. In embodiments, the weight ratio of nalbuphine to hydrophilic compound is about 10:1 to about 1:1, about 10:1 to about 2:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, or about 2:1 to about 1:1, including any values or ranges therebetween. In embodiments, the weight ratio of nalbuphine to hydrophilic compound is about 6:1 to about 1:1, about 5:1 to about 2:1, about 4:1 to about 3:1, about 4:1 to about 2:1, or about 5:1 to about 2:1, including any values or ranges therebetween. In embodiments, the weight ratio of nalbuphine to hydrophilic compound is about 1:5, about 1:4.5, about 1:4.4, about 1:4, about 1:3.5, about 1:3.3, about 1:3, about 1:2.5, about 1:2, about 1:1, or about 1:1.5, including any values or ranges therebetween.

The sustained release delivery system comprises the hydrophilic compound in an amount of about 5% to about 80% by weight. In embodiments, the sustained release delivery system comprises the hydrophilic compound in an amount of about 5% to about 30%, about 8% to about 31%, about 10% to about 20%, about 20% to about 60%, or about 40% to about 60% by weight, including any values or ranges therebetween. In embodiments, the sustained release delivery system comprises the hydrophilic compound in an amount of about 8% to about 31% by weight. In embodiments, the sustained release delivery system comprises the hydrophilic compound in an amount of about 10% to about 20% by weight. In embodiments, the sustained release delivery system comprises the hydrophilic compound in an amount of about 10%, about 11%, about 12%, about 13%, about 14%, about 15%, about 16%, about 17%, about 18%, about 19%, or about 20% by weight. In embodiments, the sustained release delivery system comprises the hydrophilic compound in an amount of about 12% by weight. In embodiments, the sustained release delivery system comprises the hydrophilic compound in an amount of about 8% by weight. In embodiments, the sustained release delivery system comprises the hydrophilic compound in an amount of about 20% by weight. In embodiments, the sustained release delivery system comprises the hydrophilic compound in an amount of about 28% by weight.

The hydrophilic compound is any pharmaceutically acceptable compound known in the art to be hydrophilic. In embodiments, the hydrophilic compounds comprise pharmaceutically acceptable gums, cellulose ethers, polyvinyl pyrrolidone, protein-derived compounds, or a mixture thereof. In embodiments, the hydrophilic compound is heteropolysaccharide gum, homopolysaccharide gum, xanthan, tragacanth, pectins, acacia, karaya, alginates, agar, guar, hydroxypropyl guar, carrageenan, locust bean gum, or gellan gum, or a mixture thereof. In embodiments, the cellulose ether is hydroxyalkyl cellulose or carboxyalkyl cellulose. In embodiments, cellulose ether comprises hydroxyethyl celluloses, hydroxypropyl celluloses, hydroxypropylmethyl-celluloses, carboxy methylcelluloses, or a mixture thereof. In embodiments, the hydrophilic compound is a gum. In embodiments, the hydrophilic compound is a heteropolysaccharide gum. In embodiments, the hydrophilic compound is a xanthan gum or derivative thereof. Derivatives of xanthan gum include without limitation, for example, deacylated xanthan gum, the carboxymethyl esters of xanthan gum, and the propylene glycol esters of xanthan gum.

In embodiments, the sustained release delivery system further comprises at least one cross-linking agent. In embodiments, the cross-linking agent is a compound that is capable of cross-linking the hydrophilic compound to form a gel matrix in the presence of liquids. As used herein, “liquids” includes, for example, gastrointestinal fluids and aqueous solutions, such as those used for in vitro dissolution testing. In embodiments, the sustained release delivery system comprises the cross-linking agent in an amount of about 0.5% to about 80% by weight. In embodiments, the sustained release delivery system comprises the cross-linking agent in an amount of about 12% to about 47% by weight. In embodiments, the sustained release delivery system comprises the cross-linking agent in an amount of about 20% to about 30% by weight. In embodiments, the sustained release delivery system comprises the cross-linking agent in an amount of about 15% to about 25% by weight. In embodiments, the at least one cross-linking agent is present in the sustained release delivery system in an amount of about 15%, about 16%, about 17%, about 18%, about 19%, about 20%, about 21%, about 22%, about 23%, about 24%, or about 25% by weight, including any values or ranges therebetween. In embodiments, the sustained release delivery system comprises the cross-linking agent in an amount of about 18% by weight. In embodiments, the sustained release delivery system comprises the cross-linking agent in an amount of about 12% by weight. In embodiments, the sustained release delivery system comprises the cross-linking agent in an amount of about 30% by weight. In embodiments, the sustained release delivery system comprises the cross-linking agent in an amount of about 42% by weight.

In embodiments, the cross-linking agents are homopolysaccharides. In embodiments, the homopolysaccharide is galactomannan gum, guar gum, hydroxypropyl guar gum, or locust bean gum. In embodiments, the cross-linking agent is a locust bean gum or a guar gum. In embodiments, the cross-linking agent is an alginic acid derivative or hydrocolloid.

In embodiments, the sustained release delivery system comprises at least one hydrophilic compound and at least one cross-linking agent, the weight ratio of hydrophilic compound to cross-linking agent is about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, or about 1:2 to about 2:1, including any values or ranges therebetween. In embodiments, the weight ratio of hydrophilic compound to cross-linking agent is about 1:5, about 1:4.5, about 1:4, about 1:3.5, about 1:3, about 1:2.5, about 1:2, about 1:1.5, or about 1:1, including any values or ranges therebetween.

In embodiments, the sustained release delivery system comprises at least one hydrophilic compound and at least one cross-linking agent, the weight ratio of the nalbuphine or pharmaceutically acceptable salt, solvate or ester thereof to the sum of the at least one hydrophilic compound and the at least one cross-linking agent is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including any values or ranges therebetween. In embodiments, the weight ratio of the nalbuphine or pharmaceutically acceptable salt, solvate or ester thereof to the sum of the at least one hydrophilic compound and the at least one cross-linking agent is about 4:1 to about 1:1, about 4:1 to about 1:1.5, about 3:1 to about 1:1, or about 2:1 to about 1:1, including any values or ranges therebetween. In embodiments, the ratio of the nalbuphine or pharmaceutically acceptable salt, solvate or ester thereof to the sum of the at least one hydrophilic compound and the at least one cross-linking agent is about 5:1, about 4:1 (i.e., 1:0.25), about 3.5:1, about 3:1, about 2.5:1, about 2:1 (i.e., 1:0.5), about 1.9:1, about 1.8:1, about 1.7:1, about 1.6:1, about 1.5:1, about 1.4:1, about 1.3:1, about 1.2:1, about 1.1:1, about 1:1, about 1:1.5, about 1:2, about 1:3, about 1:4, or about 1:5, including any values or ranges therebetween.

In embodiments, the sustained release delivery system further comprises one or more pharmaceutical diluents known in the art. In embodiments, the pharmaceutical diluent is monosaccharide, disaccharide, polyhydric alcohol, or a mixture thereof. In embodiments, pharmaceutical diluent is starch, mannitol, lactose, dextrose, sucrose, microcrystalline cellulose, sorbitol, xylitol, fructose, or a mixture thereof. In embodiments, the pharmaceutical diluent is water-soluble. Nonlimiting examples of water-soluble pharmaceutical diluents include lactose, dextrose, sucrose, or a mixture thereof. The weight ratio of pharmaceutical diluent to hydrophilic compound is about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, or about 1:2 to about 2:1, including any values or ranges therebetween. In embodiments, the weight ratio of pharmaceutical diluent to hydrophilic compound is about 9:1 to about 1:1.5. In embodiments, the weight ratio of pharmaceutical diluent to hydrophilic compound is about 9:1, about 8.75:1, about 8.5:1, about 8.25:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1, including any values or ranges therebetween.

In embodiments, the sustained release delivery system comprises one or more pharmaceutical diluents in an amount of about 20% to about 80%, about 30% to about 70%, about 40% to about 70%, or about 40% to about 60%. In embodiments, the sustained release delivery system comprises one or more pharmaceutical diluents in an amount of about 20% to about 70% by weight. In embodiments, the sustained release delivery system comprises one or more pharmaceutical diluents in an amount of about 50% to about 85% by weight. In embodiments, the sustained release delivery system comprises one or more pharmaceutical diluents in an amount of about 55%, about 60%, about 65%, about 70%, about 80%, or about 85% by weight. In embodiments, the sustained release delivery system comprises one or more pharmaceutical diluents in an amount of about 20% by weight. In embodiments, the sustained release delivery system comprises one or more pharmaceutical diluents in an amount of about 30% by weight. In embodiments, the sustained release delivery system comprises one or more pharmaceutical diluents in an amount of about 40% by weight. In embodiments, the sustained release delivery system comprises one or more pharmaceutical diluents in an amount of about 50% by weight. In embodiments, the sustained release delivery system comprises one or more pharmaceutical diluents in an amount of about 60% by weight. In embodiments, the sustained release delivery system comprises one or more pharmaceutical diluents in an amount of about 70% by weight.

In embodiments, the sustained release delivery system comprises one or more cationic cross-linking compounds. In embodiments, the one or more cationic cross-linking compounds are used instead of the cross-linking agent. In embodiments, the one or more cationic cross-linking compounds are used in addition to the cross-linking agent. In embodiments, the one or more cationic cross-linking compounds are used in an amount sufficient to cross-link the hydrophilic compound to form a gel matrix in the presence of liquids. In embodiments, the one or more cationic cross-linking compounds are present in the sustained release delivery system in an amount of about 0.5% to about 30%, about 0.5% to about 25%, about 0.5% to about 20%, about 0.5% to about 15%, about 0.5% to about 10%, or about 0.5% to about 5% by weight, including any values or ranges therebetween. In embodiments, the one or more cationic cross-linking compounds are present in the sustained release delivery system in an amount of about 5% to about 20%, about 5% to about 15%, about 6% to about 14%, about 7% to about 13%, about 8% to about 12%, or about 9% to about 11% by weight, including any values or ranges therebetween. In embodiments, the one or more cationic cross-linking compounds are present in the sustained release delivery system in an amount of about 5%, about 6%, about 7%, about 8%, about 9%, about 10%, about 11%, about 12%, about 13%, about 14%, or about 15% by weight, including any values or ranges therebetween. In embodiments, the cationic cross-linking compound is present in the sustained release delivery system in an amount of about 10% by weight.

In embodiments, the cationic cross-linking compound is monovalent metal cation, multivalent metal cation, and inorganic salt, including alkali metal and/or alkaline earth metal sulfate, chloride, borate, bromide, citrate, acetate, lactate, or a mixture thereof. In embodiments, the cationic cross-linking compound comprises one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or a mixture thereof.

In embodiments, the sustained release delivery system comprises at least one hydrophilic compound and at least one cationic cross-linking compound, the weight ratio of hydrophilic compound to cationic cross-linking compound is about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1, including any values or ranges therebetween. In embodiments, the weight ratio of hydrophilic compound to cationic cross-linking compound is about 1:3 to about 3:1. In embodiments, the weight ratio of hydrophilic compound to cationic cross-linking compound is about 3:1, about 2.75:1, about 2.5:1, about 2.25:1, about 2:1, about 1.8:1, about 1.6:1, about 1.4:1, about 1.2:1, about 1:1, about 1:1.25, about 1:1.5, or about 1:2, including any values or ranges therebetween. In embodiments, the weight ratio of hydrophilic compound to cationic cross-linking compound is about 1:1.25. In embodiments, the weight ratio of hydrophilic compound to cationic cross-linking compound is about 1.2:1. In embodiments, the weight ratio of hydrophilic compound to cationic cross-linking compound is about 2:1. In embodiments, the weight ratio of hydrophilic compound to cationic cross-linking compound is about 2.8:1.

In embodiments, the at least one hydrophilic compound is present in the sustained release delivery system in an amount of about 5% to about 80% by weight; the at least one cationic cross-linking agent is present in the sustained release delivery system in an amount of about 0.5% to about 30% by weight; and the at least one pharmaceutical diluent is present in the sustained release delivery system in an amount of about 20% to about 80% by weight. In embodiments, the at least one hydrophilic compound is present in the sustained release delivery system in an amount of about 8% to about 30% by weight; the at least one cationic cross-linking agent is present in the sustained release delivery system in an amount of about 10% by weight; and the at least one pharmaceutical diluent is present in the sustained release delivery system in an amount of about 20% to about 70% by weight. In embodiments, the at least one hydrophilic compound is present in the sustained release delivery system in an amount of about 5% to about 30% by weight; the at least one cationic cross-linking agent is present in the sustained release delivery system in an amount of about 5% to about 20% by weight; and the at least one pharmaceutical diluent is present in the sustained release delivery system in an amount of about 20% to about 85% by weight. In embodiments, the at least one hydrophilic compound is present in the sustained release delivery system in an amount of about 10% to about 20% by weight; the at least one cationic cross-linking agent is present in the sustained release delivery system in an amount of about 5% to about 15% by weight; and the at least one pharmaceutical diluent is present in the sustained release delivery system in an amount of about 50% to about 85% by weight.

In embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, or about 30% by weight, including any values or ranges therebetween; the at least one cationic cross-linking agent is present in the sustained release delivery system in an amount of about 5%, about 6%, about 7%, about 8%, about 9%, about 10%, about 11%, about 12%, about 13%, about 14%, about 15%, about 16%, about 17%, about 18%, about 19%, or about 20%, by weight, including any values or ranges therebetween; and the at least one pharmaceutical diluent is present in the sustained release delivery system in an amount of about 40%, about 45%, about 50%, about 55%, about 60%, about 65%, about 70%, about 80%, or about 85% by weight, including any values or ranges therebetween. In embodiments, the at least one hydrophilic compound is present in the sustained release delivery system in an amount of about 10%, about 11%, about 12%, about 13%, about 14%, about 15%, about 16%, about 17%, about 18%, about 19%, or about 20% by weight, including any values or ranges therebetween; the at least one cationic cross-linking agent is present in the sustained release delivery system in an amount of about 5%, about 6%, about 7%, about 8%, about 9%, about 10%, about 11%, about 12%, about 13%, about 14%, about 15%, by weight, including any values or ranges therebetween; and the at least one pharmaceutical diluent is present in the sustained release delivery system in an amount of about 55%, about 60%, about 65%, about 70%, about 80%, or about 85% by weight, including any values or ranges therebetween. In embodiments, the at least one hydrophilic compound is present in the sustained release delivery system in an amount of about 8%, about 12%, or about 20% by weight; the at least one cationic cross-linking agent is present in the sustained release delivery system in an amount of about 10%, about 12%, or about 14% by weight; and the at least one pharmaceutical diluent is present in the sustained release delivery system in an amount of about 40%, about 60%, or about 70% by weight.

In embodiments, the sustained release delivery system comprises about 0.5% to about 80% locust bean gum, about 5% to about 80% xanthan gum, about 20% to about 80% mannitol and about 0.5% to 80% calcium sulfate dihydrate. In embodiments, the sustained release delivery system comprises about 12% to about 47% locust bean gum, about 8% to about 31% xanthan gum, about 20% to about 78% mannitol and about 0.5% to 25% calcium sulfate dihydrate. In embodiments, the sustained release delivery system comprises about 15% to about 25% locust bean gum, about 10% to about 20% xanthan gum, about 50% to about 85% mannitol and about 5% to 15% calcium sulfate dihydrate. In embodiments, the sustained release delivery system comprises about 18% locust bean gum, about 12% xanthan gum, about 60% mannitol and about 10% calcium sulfate dihydrate. In embodiments, the sustained release delivery system comprises about 12% locust bean gum, about 8% xanthan gum, about 70% mannitol and about 10% calcium sulfate dihydrate. In embodiments, the sustained release delivery system comprises about 20% locust bean gum, about 30% xanthan gum, about 40% mannitol and about 10% calcium sulfate dihydrate. In embodiments, the sustained release delivery system comprises about 30% locust bean gum, about 20% xanthan gum, about 40% mannitol and about 10% calcium sulfate dihydrate. In embodiments, the sustained release delivery system comprises about 42% locust bean gum, about 28% xanthan gum, about 20% mannitol and about 10% calcium sulfate dihydrate.

In embodiments, two properties of the components of this sustained release system (e.g., the at least one hydrophilic compound and the at least one cross-linking agent; or the at least one hydrophilic compound and at least one cationic cross-linking compound) are that it forms a gel matrix upon exposure to liquids are fast hydration of the compounds/agents and the ability to form a gel matrix having a high gel strength. These two properties, which are needed to achieve a slow release gel matrix, are maximized by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby acts synergistically to provide a higher than expected viscosity (i.e., high gel strength) of the gel matrix.

In embodiments, the sustained release compositions are further admixed with one or more wetting agents (e.g., polyethoxylated castor oil, polyethoxylated hydrogenated castor oil, polyethoxylated fatty acid from castor oil, polyethoxylated fatty acid from hydrogenated castor oil) one or more lubricants (e.g., magnesium stearate, sodium stearyl fumarate, and the like), one or more buffering agents, one or more colorants, and/or other conventional ingredients.

In embodiments, compositions employed in the present methods can contain additional pharmaceutical excipients. For example, In embodiments, fumaric acid can be added to the formulations described herein.

In embodiments, a non-functional coating, e.g., Opadry® can be added to the compositions described herein.

In embodiments, the compositions described herein further include a second hydrophilic compound. In embodiments, the second hydrophilic compound is a cellulose ether. In embodiments, the second hydrophilic compound is a hydroxyalkyl cellulose or a carboxyalkyl cellulose. In embodiments, the second hydrophilic compound is a hydroxyethyl cellulose, a hydroxypropyl cellulose, a hydroxypropylmethyl-cellulose, a carboxy methylcellulose, or a mixture thereof. In embodiments, the second hydrophilic is an ethyl cellulose or wax (e.g., including without limitation cetyl alcohol, stearyl alcohol, white wax, or carnauba wax). The second hydrophilic compound is present in the formulation in an amount ranging from about 5% to about 45%, about 5% to about 25%, about 10% to about 20%, or 12% to about 18% by weight. In embodiments, the second hydrophilic compound is present in the formulation in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, or about 45%, including any values or ranges therebetween.

In embodiments, the weight ratio of the second hydrophilic compound to the nalbuphine or pharmaceutically acceptable salt, solvate or ester is about 5:1 to about 1:5, about 4:1 to about 1:4, about 3:1 to about 1:3, about 2:1 to about 1:2, about 1:1 to about 1:3, or about 1:1 to about 1:2, including any values or ranges therebetween. In embodiments, the weight ratio of the second hydrophilic compound to the nalbuphine or pharmaceutically acceptable salt, solvate or ester is about 5:1, about 4:1, about 3:1, about 2:1, about 1:1, about 1:2, about 1:3, about 1:4, or about 1:5, including any values or ranges therebetween.

In embodiments, the weight ratio of the second hydrophilic compound to the sustained release delivery system is about 10:1 to about 1:10, about 8:1 to about 1:8, about 6:1 to about 1:6, about 4:1 to about 1:4, about 2:1 to about 1:3, about 1:1 to about 1:10, about 1:1 to about 1:6, or about 1:2 to about 1:6, including any values or ranges therebetween. In embodiments, the weight ratio of the second hydrophilic compound to the sustained release delivery system is about 10:1, about 8:1, about 6:1, about 4:1, about 2:1, about 1:1, about 1:1.5, about 1:2, about 1:2.5, about 1:3, about 1:4, about 1:5, about 1:6, about 1:7, about 1:8, about 1:9 or about 1:10, including any values or ranges therebetween.

In embodiments, the oral sustained release solid dosage formulations including from about 1 mg to 200 mg nalbuphine hydrochloride and about 10 mg to about 420 mg of a sustained release delivery system. In embodiments, the sustained release delivery system comprises about 12% to about 42% locust bean gum; about 8.0% to about 28% xanthan gum; about 20% to about 70% mannitol; and about 5% to about 20% calcium sulfate dihydrate. In embodiments, the present methods can employ oral sustained release solid dosage formulations including from about 5 mg to about 80 mg nalbuphine hydrochloride and about 80 mg to about 360 mg of a sustained release delivery system. In embodiments, the present methods can employ oral sustained release solid dosage formulations including from about 50 mg to about 150 mg nalbuphine hydrochloride and about 100 mg to about 300 mg of a sustained release delivery system.

In embodiments, the present methods employ oral sustained release solid dosage formulations including about 15 mg nalbuphine hydrochloride, and from about 25 mg to about 225 mg, for example about 195 mg, of a sustained release delivery system. In embodiments, the sustained release delivery system comprises about 14% locust bean gum; about 9% xanthan gum; about 47% mannitol; and about 8% calcium sulfate dihydrate.

In embodiments, the present methods employ oral sustained release solid dosage formulations including about 30 mg nalbuphine hydrochloride, and from about 25 mg to about 225 mg, for example about 180 mg, of a sustained release delivery system. In embodiments, the sustained release delivery system comprises about 18% locust bean gum; about 12% xanthan gum; about 60% mannitol; and about 10% calcium sulfate dihydrate.

In embodiments, the present methods employ oral sustained release solid dosage formulations including about 60 mg nalbuphine hydrochloride, and from about 25 mg to about 225 mg, for example about 120 mg, of a sustained release delivery system. In embodiments, the sustained release delivery system comprises about 10% locust bean gum; about 12% xanthan gum; about 60% mannitol; and about 10% calcium sulfate dihydrate. In embodiments, the present methods employ oral sustained release solid dosage formulations including from about 5 mg to about 80 mg nalbuphine hydrochloride and about 80 mg to about 360 mg of a sustained release delivery system.

In embodiments, the present methods employ oral sustained release solid dosage formulations including about 120 mg nalbuphine hydrochloride, and from about 25 mg to about 250 mg, for example about 240 mg of a sustained release delivery system. In embodiments, the sustained release delivery system comprises about 18% locust bean gum; about 12% xanthan gum; about 60% mannitol; and about 10% calcium sulfate dihydrate.

In embodiments, the present methods employ oral sustained release solid dosage formulations including about 30 mg nalbuphine hydrochloride, and from about 25 mg to about 350 mg, for example about 270 mg or about 360 mg of a sustained release delivery system. In embodiments, the sustained release delivery system comprises about 18% locust bean gum; about 12% xanthan gum; about 60% mannitol; and about 10% calcium sulfate dihydrate.

In embodiments, the present methods employ oral sustained release solid dosage formulations including about 45 to about 60 mg nalbuphine hydrochloride and from about 100 mg to about 200 mg of a sustained release delivery system. In embodiments, the sustained release delivery system comprises about 15% to about 25% locust bean gum; about 10% to about 20% xanthan gum; about 50% to about 85% mannitol; and about 5% to about 15% calcium sulfate dihydrate.

In embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, and about 1.9 mg magnesium stearate.

In embodiments, the present methods employ oral sustained release solid dosage formulations including about 29.8 mg nalbuphine hydrochloride, about 32.2 mg locust bean gum; about 21.4 mg xanthan gum; about 107 mg mannitol; about 18 mg calcium sulfate dihydrate, about 35 mg hydroxypropylcellulose, and about 1.9 mg magnesium stearate.

In embodiments, the present methods employ oral sustained release solid dosage formulations including about 60 mg nalbuphine hydrochloride, about 21.6 mg locust bean gum; about 14.4 mg xanthan gum; about 72 mg mannitol; about 12 mg calcium sulfate dihydrate, about 30 mg hydroxypropylcellulose, and about 1.6 mg magnesium stearate.

In embodiments, the present methods employ oral sustained release solid dosage formulations including about 59.5 mg nalbuphine hydrochloride, about 21.4 mg locust bean gum; about 14.3 mg xanthan gum; about 71 mg mannitol; about 12 mg calcium sulfate dihydrate, about 30 mg hydroxypropylcellulose, and about 1.6 mg magnesium stearate.

In embodiments, the present methods employ oral sustained release solid dosage formulations including about 120 mg nalbuphine hydrochloride, about 43.2 mg locust bean gum; about 28.8 mg xanthan gum; about 144 mg mannitol; about 24 mg calcium sulfate dihydrate, about 60 mg hydroxypropylcellulose, and about 3.2 mg magnesium stearate.

In embodiments, the present methods employ oral sustained release solid dosage formulations including about 119.0 mg nalbuphine hydrochloride, about 42.9 mg locust bean gum; about 25.6 mg xanthan gum; about 143 mg mannitol; about 24 mg calcium sulfate dihydrate, about 60 mg hydroxypropylcellulose, and about 3 mg magnesium stearate.

In embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, about 1.9 mg magnesium stearate, and about 7.4 mg Opadry II White.

In embodiments, the oral sustained release solid dosage formulations including from about 1 mg to 170 mg nalbuphine hydrochloride and about 10 mg to about 420 mg of a sustained release delivery system. In embodiments, the sustained release delivery system comprises about 12% to about 42% locust bean gum; about 8.0% to about 28% xanthan gum; about 20% to about 70% mannitol; and about 5% to about 20% calcium sulfate dihydrate. In embodiments, the present methods can employ oral sustained release solid dosage formulations including from about 5 mg to about 80 mg nalbuphine hydrochloride and about 80 mg to about 360 mg of a sustained release delivery system. In embodiments, the present methods can employ oral sustained release solid dosage formulations including from about 50 mg to about 150 mg nalbuphine hydrochloride and about 100 mg to about 300 mg of a sustained release delivery.

The sustained release formulations of nalbuphine are orally administrable solid dosage formulations. In embodiments, the oral solid dosage formulations comprise tablets, capsules including a plurality of granules, sublingual tablets, powders, granules, syrups, and buccal dosage forms or devices (e.g., buccal patches, tablets, etc.). In embodiments, tablets have an enteric coating or a hydrophilic coating.

In embodiments, the sustained release delivery system is prepared by dry granulation or wet granulation, before the nalbuphine, or pharmaceutically acceptable salt, solvate or ester thereof, is added, although the components can be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the nalbuphine, or the pharmaceutically acceptable salt, solvate or ester thereof, and, optionally, one or more wetting agents, one or more lubricants, one or more buffering agents, one or more coloring agents, one or more second hydrophilic compounds, or other conventional ingredients, to produce a granulated composition. In embodiments, the sustained release delivery system and the nalbuphine are blended with a high shear mixer. In embodiments, the nalbuphine is finely and homogeneously dispersed in the sustained release delivery system. In embodiments, the granulated composition, in an amount sufficient to make a uniform batch of tablets, is subjected to tableting in a conventional production scale tableting machine at typical compression pressures, i.e., about 2,000-16,000 psi. In embodiments, the mixture is not compressed to a point where there is subsequent difficulty with hydration upon exposure to liquids.

In embodiments, the nalbuphine formulation is prepared by dry granulation or wet granulation. In embodiments, the components of the sustained release delivery system are added, along with the nalbuphine, or a pharmaceutically acceptable salt, solvate or ester thereof. In embodiments, all of the components are held together by an agglomeration technique to produce an acceptable product. In the wet granulation technique, nalbuphine, or pharmaceutically salt, solvate or ester thereof, and the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules.

In embodiments, one or more wetting agents, one or more lubricants, one or more buffering agents, one or more coloring agents, one or more second hydrophilic compounds, or other conventional ingredients, are also added to the granulation. The granulated composition, in an amount sufficient to make a uniform batch of tablets, is subjected to tableting in a conventional production scale tableting machine at typical compression pressures, i.e., about 2,000-16,000 psi. In embodiments, the mixture should not be compressed to a point where there is subsequent difficulty with hydration upon exposure to liquids.

In embodiments, the average particle size of the granulated composition is from about 50 μm to about 400 μm by weight. In embodiments, the average particle size by weight is from about 185 μm to about 265 μm. In embodiments, the average density of the granulated composition is from about 0.3 g/mL to about 0.8 g/mL. In embodiments, the average density is from about 0.5 g/mL to about 0.7 g/mL. In embodiments, the tablets formed from the granulations are generally from about 4 kp to about 22 kp hardness. The average flow of the granulations is from about 25 to about 40 g/sec.

In embodiments, the present methods employ a multilayer solid dosage form, in which the layers are formulated to release the nalbuphine hydrochloride at different rates. In embodiments, the second layer is an extended release layer that comprises nalbuphine or a pharmaceutically acceptable salt, solvate or ester thereof and a sustained release delivery system designed to release the nalbuphine or the pharmaceutically acceptable salt, solvate or ester thereof at a controlled rate so that therapeutically effective blood levels are maintained over an extended period of time (e.g., from about 8 to about 12 hours). In embodiments, the first layer is an immediate release layer that comprises a formulation of nalbuphine or a pharmaceutically acceptable salt, solvate or ester thereof designed to release the nalbuphine or the pharmaceutically acceptable salt, solvate or ester thereof at a rate that is faster than the rate of the second layer to achieve a therapeutically effective blood level in an immediate period of time (e.g., from about 1 to about 2 hours). In embodiments, the first layer comprises a sustained release delivery system. In embodiments, the first layer does not include a sustained release delivery system.

In embodiments, the weight ratio of the second layer to the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including any values or ranges therebetween. In embodiments, the weight ratio of the second layer to the first layer is about 5:1 to about 1:5. In embodiments, the weight ratio of the second layer to the first layer is about 1:1 to about 1:2. In embodiments, the weight ratio of the second layer to the first layer is about 1:1, about 1:1.2, about 1:1.4, about 1:1.6, about 1:1.8, or about 1:2. In embodiments, the weight ratio of the second layer to the first layer is about 1:2. In embodiments, the weight ratio of the second layer to the first layer is about 1:1.4. In embodiments, the weight ratio of the second layer to the first layer is about 3:1, about 2.5:1, about 2:1, about 1.5:1. In embodiments, the weight ratio of the second layer to the first layer is about 2.5:1.

In embodiments, the sustained release delivery system of the multilayer dosage form comprises (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. In embodiments, when the first layer comprises a sustained release delivery system, the sustained release delivery system of the first layer comprises the same components as the sustained release delivery system of the second layer (e.g., both the first and second layers are one of embodiments (i)-(iii), listed above). In embodiments, the sustained release delivery system of the first layer comprises different components as the sustained release delivery system of the second layer (e.g., the first layer is embodiment (i), listed above, while the second layer is embodiment (iii), listed above). It is recognized that the sustained release delivery system of either layer is one of embodiments (i)-(iii) listed above. Moreover, it is recognized that, in embodiments, the first layer does not include a sustained release delivery system.

In embodiments, the sustained release delivery system is present in the second layer (e.g., extended release layer) in an amount of about 10 mg to about 420 mg. In embodiments, the sustained release delivery system is in the second layer in an amount of about 110 mg to about 200 mg. In embodiments, the sustained release delivery system is present in the second layer in an amount of about 110 mg to about 150 mg. In embodiments, the sustained release delivery system is present in the second layer in an amount of about 90 mg to about 150 mg. In embodiments, the sustained release delivery system is present in the second layer in an amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg, including any values or ranges therebetween. In embodiments, the sustained release delivery system is present in the second layer in an amount of about 123 mg. In embodiments, the sustained release delivery system is present in the second layer in an amount of about 101 mg. In embodiments, the sustained release delivery system is present in the second layer in an amount of about 92 mg. In embodiments, the sustained release delivery system is present in the second layer in an amount of about 112.5 mg. In embodiments, the sustained release delivery system is present in the second layer in an amount of about 135 mg. In embodiments, the sustained release delivery system is present in the second layer in an amount of about 150 mg.

In embodiments, nalbuphine or a pharmaceutically acceptable salt, solvate or ester thereof is present in the second layer in an amount of about 15 mg to about 60 mg. In embodiments, nalbuphine or a pharmaceutically acceptable salt, solvate or ester thereof is present in the second layer in an amount of about 30 mg to about 60 mg. In embodiments, nalbuphine or a pharmaceutically acceptable salt, solvate or ester thereof is present in the second layer in an amount of about 45 mg to about 60 mg. In embodiments, nalbuphine or a pharmaceutically acceptable salt, solvate or ester thereof is present in the second layer in an amount of about 15 mg. In embodiments, nalbuphine or a pharmaceutically acceptable salt, solvate or ester thereof is present in the second layer in an amount of about 30 mg. In embodiments, nalbuphine or a pharmaceutically acceptable salt, solvate or ester thereof is present in the second layer in an amount of about 45 mg. In embodiments, nalbuphine or a pharmaceutically acceptable salt, solvate or ester thereof is present in the second layer in an amount of about 15 mg, about 30 mg, about 60 mg, about 90 mg, about 120 mg, or about 180 mg, including any values or ranges therebetween.

In embodiments, the weight ratio of nalbuphine or a pharmaceutically acceptable salt, solvate or ester thereof to the sustained release delivery system in the second layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including any values or ranges therebetween. In embodiments, the weight ratio of nalbuphine or a pharmaceutically acceptable salt, solvate or ester thereof to the sustained release delivery system in the second layer is about 1:2 to about 1:4. In embodiments, the weight ratio of nalbuphine or a pharmaceutically acceptable salt, solvate or ester thereof to the sustained release delivery system in the second layer is about 1:1 to about 1:5. In embodiments, the weight ratio of nalbuphine or a pharmaceutically acceptable salt, solvate or ester thereof to the sustained release delivery system in the second layer is about 1:1, about 1:1.2, about 1:1.4, about 1:1.6, about 1:1.8, about 1:2, about 1:2.5, about 1:3, or about 1:3.5, including any values or ranges therebetween. In embodiments, the weight ratio of nalbuphine or a pharmaceutically acceptable salt, solvate or ester thereof to the sustained release delivery system in the second layer is about 1:2.5. In embodiments, the weight ratio of nalbuphine or a pharmaceutically acceptable salt, solvate or ester thereof to the sustained release delivery system in the second layer is about 1:3.3. In a further embodiment, the weight ratio of nalbuphine or a pharmaceutically acceptable salt, solvate or ester thereof to the sustained release delivery system in the second layer is about 1:3. In embodiments, the ratio of nalbuphine or a pharmaceutically acceptable salt, solvate or ester thereof to the sustained release delivery system in the second layer is about 1:2.

In embodiments, the sustained release delivery system is present in the first layer (e.g., immediate release layer) at an amount of about 0 mg to about 50 mg. In embodiments, the sustained release delivery system is present in the first layer in an amount of about 5 mg to about 25 mg or about 5 mg to about 15 mg. In embodiments, the sustained release delivery system is present in the first layer in an amount of about 3 mg to about 9 mg. In embodiments, the sustained release delivery system is present in the first layer in an amount of about 4 mg to about 6 mg. In embodiments, the sustained release delivery system is present in the first layer in an amount of about 2 mg, about 4 mg, about 6 mg, about 8 mg, about 10 mg, about 12 mg, about 14 mg, about 15 mg, about 16 mg, about 18 mg, about 20 mg about 25 mg, about 30 mg, about 35 mg, about 40 mg, about 45 mg or about 50 mg, including any values or ranges therebetween. In embodiments, the sustained release delivery system is present in the first layer in an amount of about 6 mg.

In embodiments, nalbuphine or a pharmaceutically acceptable salt, solvate or ester thereof is generally present in the first layer (e.g., immediate release layer) in an amount of about 5 mg to about 180 mg. In embodiments, nalbuphine or a pharmaceutically acceptable salt, solvate or ester thereof is present in the first layer in an amount ranging from about 5 mg to about 25 mg or from about 10 mg to about 20 mg. In embodiments, the nalbuphine or a pharmaceutically acceptable salt, solvate or ester thereof is present in the first layer in an amount of about 5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg or about 50 mg, including any values or ranges therebetween. In embodiments, nalbuphine or a pharmaceutically acceptable salt, solvate or ester thereof is present in the first layer in an amount of about 15 mg, about 30 mg, about 60 mg, about 90 mg, about 120 mg, or about 180 mg, including any values or ranges therebetween.

In embodiments, the first layer comprises a sustained release delivery system and the ratio of nalbuphine or pharmaceutically acceptable salt, solvate or ester thereof to the sustained release delivery system in the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2, including any values or ranges therebetween. In embodiments, the ratio of nalbuphine or pharmaceutically acceptable salt, solvate or ester thereof to the sustained release delivery system in the first layer is about 2:1 to about 4:1. In embodiments, the ratio of nalbuphine or pharmaceutically acceptable salt, solvate or ester thereof to the sustained release delivery system in the first layer is about 5:1, about 4.5:1, about 4:1, about 3.5:1, about 3:1, about 2.5:1, about 2:1, about 1.5:1, or about 1:1, including any values or ranges therebetween. In embodiments, the ratio of nalbuphine or pharmaceutically acceptable salt, solvate or ester thereof to the sustained release delivery system in the first layer is about 2.5:1. In another embodiment, the ratio of nalbuphine or pharmaceutically acceptable salt, solvate or ester thereof to the sustained release delivery system in the first layer is about 3:1.

In embodiments, the multilayer dosage form further comprises a pharmaceutical disintegrant. The disintegrant promotes the dissolution and absorption of nalbuphine or pharmaceutically acceptable salt, solvate or ester thereof from the immediate release layer. In embodiments, the pharmaceutical disintegrant is croscarmellose sodium, starch glycolate, crospovidone, unmodified starch, or a mixture thereof. In embodiments, the disintegrant is in the first layer (i.e., the immediate release layer), of the dosage form. The disintegrant is generally present in the layer in an amount of about 1.5 mg to about 4.5 mg. In embodiments, the disintegrant is present in an amount of about 3 mg. In embodiments, the disintegrant is present in the layer in an amount of about 2-10% by weight. In embodiments, the disintegrant is present in the layer in an amount of about 5% by weight. In embodiments, the layer contains a sustained release delivery system, and the weight ratio of the sustained release delivery system to the disintegrant is in a range of about 5:1 to about 1:5. In embodiments, the ratio of the sustained release delivery system to the disintegrant is in a range of about 1:1 to about 3:1. In embodiments, the ratio of the sustained release delivery system to the disintegrant is in a range of about 2:1.

In embodiments, the multilayer tablets are prepared by first preparing the immediate release layer and extended-release layer blends separately. The extended-release layer is prepared as described above. The wet granulation of the extended-release layer is then dried and milled to an appropriate size. Magnesium stearate is added and mixed with the milled granulation. The immediate release layer is prepared by first mixing the nalbuphine or the pharmaceutically acceptable salt, solvate or ester thereof with one or more diluents (e.g., microcrystalline cellulose). This mix is then optionally mixed with one or more disintegrants. The blend is mixed with magnesium stearate. Finally, the immediate release layer blend and the extended-release layer blend are compressed into multi-layer (e.g., bi-layer) tablets.

In embodiments, the chemistry of certain of the components of the formulation, such as the hydrophilic compound (e.g., xanthan gum), is such that the components are considered to be self-buffering agents which are substantially insensitive to the solubility of the nalbuphine and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, the sustained release formulation can loosely interact with the mucin in the gastrointestinal tract and thereby provide another mode by which a constant rate of delivery of the nalbuphine is achieved.

The phenomenon discussed above (muco-adhesive properties) is a mechanism by which the sustained release formulations can interact with the mucin and fluids of the gastrointestinal tract and provide a constant rate of delivery of the nalbuphine.

When measured by USP Procedure Drug Release General Chapter <711> Dissolution, (incorporated by reference herein in its entirety), the sustained release formulations employed in the present methods generally exhibit an in vitro dissolution of about 15% to about 50% by weight nalbuphine after 1 hour, about 45% to about 80% by weight nalbuphine after 4 hours, or at least about 80% by weight nalbuphine after 10 hours. In embodiments, the in vitro and in vivo release characteristics of the sustained release formulations are modified using mixtures of one or more different water insoluble and/or water-soluble compounds, using different plasticizers, varying the thickness of the sustained release film, including providing release-modifying compounds in the coating, and/or by providing passageways through the coating. In embodiments, the dissolution rate is determined using apparatus USP Type III/250 mL at pH 6.8, 37° C. and 15 dpm. In embodiments, the dissolution rate is determined using apparatus USP Type III/250 mL performed in pH change (0-1 hours pH 1.2, after hour 1 pH 4.5, after hour 2 pH 6.8) at 37° C. and 15 dpm.

In embodiments, the sustained release formulation has an in vitro dissolution of about 50% to about 100% by weight nalbuphine after about 6 hours. In embodiments, the sustained release formulation has an in vitro dissolution of about 75% to about 100% by weight nalbuphine after about 6 hours. In embodiments, the sustained release formulation has an in vitro dissolution of about 75% to about 100% by weight nalbuphine from about 6 hours to about 8 hours. In embodiments, the sustained release formulation has an in vitro dissolution of about 80% to about 100% by weight nalbuphine after about 12 hours. In embodiments, the sustained release formulation has an in vitro dissolution of about 80% to about 100% by weight nalbuphine from about 12 hours to about 24 hours. In embodiments, the sustained release formulation has an in vitro dissolution of about 80% to about 100% after about 8 hours to about 12 hours. In embodiments, the sustained release formulation has an in vitro dissolution of about 15% to about 75% by weight nalbuphine after about 1 hour. In embodiments, the sustained release formulation has an in vitro dissolution of about 50% by weight nalbuphine after about 1 hour. In embodiments, the sustained release formulation has an in vitro dissolution of about 50% by weight nalbuphine after about 1 hour and about 75% to about 100% by weight nalbuphine from about 6 hours to about 8 hours. In embodiments, the sustained release formulation has an in vitro dissolution of about 50% by weight nalbuphine after about 1 hour and about 75% to about 100% by weight nalbuphine from about 8 hours to about 12 hours. In embodiments, the sustained release formulation has an in vitro dissolution of about 50% by weight nalbuphine after about 1 hour and about 75% to about 100% by weight nalbuphine from about 12 hours to about 24 hours. In embodiments, the sustained release formulation has an in vitro dissolution of about 50% by weight nalbuphine after about 1 hour and about 80% to about 100% by weight nalbuphine after about 12 hours.

In embodiments, the tablet is a multilayer dosage form having a first extended release layer and a second, immediate release, layer, the sustained release formulation has an in vitro dissolution of about 25% to about 75% by weight nalbuphine after about 1 hour. In embodiments, the multilayer dosage form has an in vitro dissolution of about 25% by weight nalbuphine after about 1 hour. In embodiments, the multilayer dosage form has an in vitro dissolution of about 50% by weight nalbuphine after about 1 hour. In embodiments, the multilayer dosage form has an in vitro dissolution of about 75% to about 100% nalbuphine after about 6-8 hours. In embodiments, the multilayer dosage form has an in vitro dissolution of about 75% to about 100% nalbuphine after about 8-12 hours. In embodiments, the multilayer dosage form has an in vitro dissolution of about 75% to about 100% nalbuphine after about 12-24 hours. In embodiments, the multilayer dosage form has an in vitro dissolution of about 75% to about 100% nalbuphine after about 12 hours.

In embodiments, the sustained release nalbuphine formulations provide an oral unit dosage form including nalbuphine or a pharmaceutically acceptable salt, solvate or ester thereof. In embodiments, the oral dosage form provides an anti-CRS effect over a period of at least about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours, including any values or ranges therebetween. In embodiments, the oral dosage form provides an anti-CRS effect over a period of about 6-18 hours, about 8-16 hours, about 8-12 hours, about 8 to about 24 hours, about 12 to about 24 hours, about 18 to about 24 hours, or about 8-10 hours. The oral dosage form provides an anti-CRS effect over a period of about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours, including any values or ranges therebetween.

### Methods of Treatment

The present disclosure provides methods for treating a nalbuphine-treatable disorder in an elderly patient by administering an effective amount of nalbuphine, or a pharmaceutically acceptable salt, solvate or ester thereof. An effective amount is an amount sufficient to eliminate or significantly reduce symptoms associated with a nalbuphine-treatable disorder or to alleviate those symptoms. In embodiments, formulations employed in the present methods incorporate nalbuphine, or a pharmaceutically acceptable salt, or ester thereof, for the treatment of a nalbuphine-treatable disorder in an elderly patient with a daily dose of about 40% to about 60% of the daily dose administered to a non-elderly patient treated for the same nalbuphine-treatable disorder. In embodiments, the elderly patient is administered with a daily dose of about 50% to 53% of the daily dose administered to a non-elderly patient.

In embodiments, the amount of nalbuphine administered to a patient in need thereof is in the form of a pharmaceutically acceptable salt and is expressed in terms of the Equivalent Amount of Nalbuphine Free Base provided to said patient.

In embodiments, the methods for treating (e.g., safely treating) an elderly patient with a nalbuphine-treatable disorder comprise administering a daily dose of about 3 mg to about 170 mg, about 7 mg to about 170 mg, about 14 mg to about 170 mg, about 28 mg to about 170 mg, about 41 mg to about 170 mg, about 54 mg to about 170 mg, about 67 mg to about 170 mg, about 94 mg to about 170 mg, about 108 mg to about 170 mg, or about 135 mg to about 170 mg of an Equivalent Amount of Nalbuphine Free Base, including any values and sub-ranges therebetween. In embodiments, the elderly patients with a nalbuphine-treatable disorder comprises administering a daily dose of about 28 mg to about 160 mg, about 28 mg to about 150 mg, about 28 mg to about 140 mg, 28 mg to about 130 mg, about 28 mg to about 120 mg, about 28 mg to about 110 mg, 28 mg to about 100 mg, about 28 mg to about 90 mg, about 28 mg to about 80 mg, 28 mg to about 70 mg, about 28 mg to about 60 mg, about 28 mg to about 50 mg, 28 mg to about 40 mg, or about 28 mg to about 30 mg of an Equivalent Amount of Nalbuphine Free Base, including any values and sub-ranges therebetween. In embodiments, the methods for treating a nalbuphine-treatable disorder in an elderly patient comprise administering a daily dose of about 28 mg to about 170 mg of an Equivalent Amount of nalbuphine free base.

In embodiments, the methods for treating (e.g., safely treating) an elderly patient with a nalbuphine-treatable disorder comprise administering a daily dose of about 28 mg or lower. In embodiments, methods for treating (e.g., safely treating) an elderly patient with a nalbuphine-treatable disorder comprise administering a daily dose of about 25 mg, 20 mg, 14 mg, about 12 mg, about 10 mg, about 8 mg, about 6 mg, about 4 mg, or about 2 mg of an Equivalent Amount of Nalbuphine Free Base.

In embodiments, the methods for treating an elderly patient with a nalbuphine-treatable disorder comprise administering a daily dose of about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, or about 220 mg, including any values and sub-ranges therebetween, of the Equivalent Amount of Nalbuphine Free Base. In embodiments, the methods for treating (e.g., safely treating) an elderly patient with a nalbuphine-treatable disorder comprise administering a daily dose of about 7 mg, about 14 mg, about 28 mg, about 40 mg, about 54 mg, about 67 mg, about 94 mg, about 108 mg, about 135 mg, about 162 mg, or about 170 mg of the Equivalent Amount of Nalbuphine Free Base. In embodiments, the elderly patient is administered a daily dose of about 27 mg to about 170 mg of an Equivalent Amount of Nalbuphine Free Base. In embodiments, the daily dose administered to an elderly patient is about 27 mg to about 108 mg of an Equivalent Amount of Nalbuphine Free Base. In embodiments, the elderly patient is administered a daily dose of about 27 mg to about 216 mg of an Equivalent Amount of Nalbuphine Free Base.

In embodiments, the method of treating a nalbuphine-treatable disorder in a patient in need thereof comprises administering a daily dose of about 20 mg to about 340 mg (e.g., about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 222 mg, about 224 mg, about 226 mg, about 228 mg, about 230 mg, about 232 mg, about 234 mg, about 236 mg, about 238 mg, about 240 mg, about 242 mg, about 244 mg, about 246 mg, about 248 mg, about 250 mg, about 252 mg, about 254 mg, about 256 mg, about 258 mg, about 260 mg, about 262 mg, about 264 mg, about 266 mg, about 268 mg, about 270 mg, about 272 mg, about 274 mg, about 276 mg, about 278 mg, about 280 mg, about 282 mg, about 284 mg, about 286 mg, about 288 mg, about 290 mg, about 292 mg, about 294 mg, about 296 mg, about 298 mg, about 300 mg, about 302 mg, about 304 mg, about 306 mg, about 308 mg, about 310 mg, about 312 mg, about 314 mg, about 316 mg, about 318 mg, about 320 mg, about 322 mg, about 324 mg, about 326 mg, about 328 mg, about 330 mg, about 332 mg, about 334 mg, about 336 mg, about 338 mg, about 340 mg, including any values and sub-ranges therebetween) of an Equivalent Amount of Nalbuphine Free Base. In embodiments, the daily dose administered to a non-elderly patient is about 54 mg to about 216 mg of an Equivalent Amount of Nalbuphine Free Base. In embodiments, the daily dose administered to a non-elderly patient is about 54 mg to about 324 mg of an Equivalent Amount of Nalbuphine Free Base.

In embodiments, the nalbuphine-treatable disorder is uremic pruritus and the elderly patient is administered a daily dose of about 27 mg to about 170 mg of an Equivalent Amount of Nalbuphine Free Base. In embodiments, the nalbuphine-treatable disorder is uremic pruritus and the non-elderly patient is administered a daily dose of about 54 mg to about 324 mg of an Equivalent Amount of Nalbuphine Free Base.

In embodiments, the nalbuphine-treatable disorder is uremic pruritus and the elderly patient is administered a daily dose of about 27 mg to about 170 mg of an Equivalent Amount of Nalbuphine Free Base. In embodiments, the nalbuphine-treatable disorder is uremic pruritus and the non-elderly patient is administered a daily dose of about 54 mg to about 324 mg of an Equivalent Amount of Nalbuphine Free Base.

In embodiments, the nalbuphine-treatable disorder is idiopathic pulmonary fibrosis (IPF) cough and the elderly patient is administered a daily dose of about 27 mg to about 108 mg of an Equivalent Amount of Nalbuphine Free Base. In embodiments, the nalbuphine-treatable disorder is IPF cough, and the non-elderly patient is administered a daily dose of about 54 mg to about 216 mg of an Equivalent Amount of Nalbuphine Free Base. In embodiments, the nalbuphine-treatable disorder is idiopathic pulmonary fibrosis (IPF) cough and the elderly patient is administered a daily dose of about 27 mg to about 216 mg of an Equivalent Amount of Nalbuphine Free Base. In embodiments, the nalbuphine-treatable disorder is IPF cough, and the non-elderly patient is administered a daily dose of about 54 mg to about 324 mg of an Equivalent Amount of Nalbuphine Free Base.

In embodiments, the patient is administered a daily dose of nalbuphine or a pharmaceutically acceptable salt, or ester thereof once a day. In embodiments, of about 7 mg to about 170 mg, about 14 mg to about 170 mg, about 28 mg to about 170 mg, about 33 mg to about 170 mg, about 42 mg to about 170 mg, about 42 mg to about 170 mg, about 54 mg to about 170 mg, about 67 mg to about 170 mg, about 81 mg to about 170 mg, about 85 mg to about 170 mg, about 90 mg to about 170 mg, about 100 mg to about 170 mg, about 120 mg to about 170 mg, about 140 mg to about 170 mg, about 3 mg, about 5 mg, about 7 mg, about 9 mg, about 10 mg, about 12 mg, about 14 mg, about 16 mg, about 18 mg, about 20 mg, about 22 mg, about 24 mg, about 26 mg, about 28 mg, about 30 mg, about 32 mg, 34 mg, about 36 mg, about 38 mg, about 40 mg, about 42 mg, about 44 mg, about 46 mg, about 48 mg, about 50 mg, about 52 mg, about 54 mg, about 56 mg, about 58 mg, about 60 mg, about 62 mg, about 64 mg, about 66 mg, about 68 mg, about 70 mg, about 72 mg, about 74 mg, about 76 mg, about 78 mg, about 80 mg, about 82 mg, about 84 mg, about 86 mg, about 88 mg, about 90 mg, about 100 mg, about 110 mg, about 120 mg, 130 mg, about 140 mg, about 150 mg, about 160 mg, or about 170 mg of the Equivalent Amount of Nalbuphine Free Base is administered once a day, including all sub-ranges and values therebetween. In embodiments, about 28 mg to about 170 mg of the Equivalent Amount of Nalbuphine Free Base is administered once a day.

In embodiments, the patient is administered a daily dose of nalbuphine or a pharmaceutically acceptable salt, or ester thereof twice a day. In embodiments, of about 3 mg to about 85 mg, about 7 mg to about 85 mg, about 14 mg to about 85 mg, about 27 mg to about 85 mg, about 33 mg to about 85 mg, about 42 mg to about 85 mg, about 54 mg to about 85 mg, or about 67 mg to about 85 mg, e.g. about 3 mg, about 5 mg, about 7 mg, about 9 mg, about 10 mg, about 12 mg, about 14 mg, about 16 mg, about 18 mg, about 20 mg, about 22 mg, about 24 mg, about 26 mg, about 28 mg, about 30 mg, about 32 mg, 34 mg, about 36 mg, about 38 mg, about 40 mg, about 42 mg, about 44 mg, about 46 mg, about 48 mg, about 50 mg, about 52 mg, about 54 mg, about 56 mg, about 58 mg, about 60 mg, about 62 mg, about 64 mg, about 66 mg, about 68 mg, about 70 mg, about 72 mg, about 74 mg, about 76 mg, about 78 mg, about 80 mg, about 82 mg, about 84 mg, or about 85 mg of the Equivalent Amount of Nalbuphine Free Base is administered twice a day, including all sub-ranges and values therebetween. In embodiments, about 14 mg to about 86 mg of the Equivalent Amount of Nalbuphine Free Base is administered twice a day to an elderly patient in need thereof. In embodiments, about 27 mg to about 162 mg of the Equivalent Amount of Nalbuphine Free Base is administered twice a day to a non-elderly patient in need thereof. In embodiments, about 27 mg to about 108 mg of the Equivalent Amount of Nalbuphine Free Base is administered to the elderly patient twice a day. In embodiments, about 27 mg to about 108 mg of the Equivalent Amount of Nalbuphine Free Base is administered to the non-elderly patient twice a day.

In embodiments, the methods of treating a nalbuphine-treatable disorder in an elderly patient described herein further comprises a step of titrating the dose of an Equivalent Amount of Nalbuphine Free Base for at least about one week until a steady state is achieved in the patient. In embodiments, the titration is conducted for about 2 weeks until a steady state is achieved in the patient. In embodiments, the titration is conducted for about 7 days to about 30 days until a steady state is achieved in the patient. In embodiments, the titration is conducted for about 7 days to about 21 days until a steady state is achieved in the patient. In embodiments, the titration is conducted for about 7 days to about 14 days until a steady state is achieved in the patient. In embodiments, the titration is conducted until a tolerable and therapeutically effective dose of an Equivalent Amount of Nalbuphine Free Base is achieved in the patient. In embodiments, the titration is conducted for at least about 1-14 days, including at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days, including all ranges and values therebetween.

In embodiments, the elderly patient is administered titration dose of about 40% to about 60% of the titration dose administered to a non-elderly patient treated for the same nalbuphine-treatable disorder. In embodiments, the elderly patient is administered titration dose of about 1% to about 90%, about 5% to about 80%, about 10% to about 70%, about 20% to about 60%, about 30% to about 60%, about 40% to about 60%, or about 50% to about 60% of the titration dose administered to a non-elderly patient treated for the same nalbuphine-treatable disorder. In embodiments, the elderly patient is administered titration dose of about 1% to about 60%, about 2% to about 60%, about 4% to about 60%, about 6% to about 60%, about 8% to about 60%, about 10% to about 60%, about 12% to about 60%, about 14% to about 60%, about 16% to about 60%, about 18% to about 60%, about 20% to about 60% or about 22% to about 60%, about 24% to about 60%, about 26% to about 60%, about 28% to about 60%, about 30% to about 60%, about 32% to about 60%, about 34% to about 60%, about 36% to about 60%, about 38% to about 60%, about 40% to about 60%, about 42% to about 60%, about 44% to about 60% or about 46% to about 60%, about 48% to about 60%, or about 50% to about 60% of the titration dose administered to a non-elderly patient treated for the same nalbuphine-treatable disorder, including any values and sub-ranges therebetween. In embodiments, the elderly patient is administered titration dose of about 40% to about 60% of the titration dose administered to a non-elderly patient treated for the same nalbuphine-treatable disorder. In embodiments, the elderly patient is administered titration dose of about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, or about 50% of the titration dose administered to a non-elderly patient treated for the same nalbuphine-treatable disorder.

Throughout the present disclosure, the dose titration schedules disclosed herein are generally expressed in terms of the fastest titration (i.e., the least number of days) to the maximum safe and tolerable dose. However, in embodiments, the present disclosure also contemplates embodiments wherein the rate of titration is slower than the rates disclosed in the titration schedules herein (for example, the number of days to the maximum safe and tolerable dose is increased by using smaller incremental increases in the dose escalation). For example, in embodiments, the titration rate is about 30%, about 40%, about 50%, about 60%, about 70%, about 80%, about 90% or more, of the titration rate in a titration schedule disclosed herein. In embodiments, the patient's dose is titrated to the maximum dose of a titration schedule disclosed herein. In embodiments, the patient is titrated to a therapeutically effective dose that is less than the maximum dose of a disclosed titration schedule. For example, in embodiments, the patient's titration period ends on Day 4, Day 5, Day 6, Day 7, Day 8, Day 9, Day 10, Day 11, Day 12, or Day 13 because the patient achieves a safe and effective dose.

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 3 |
| Day 2 | 0 | 3 |
| Day 3 | 3 | 3 |
| Day 4 | 3 | 3 |
| Day 5 | 3 | 7 |
| Day 6 | 7 | 7 |
| Day 7 | 7 | 7 |
| Day 8 | 7 | 14 |
| Day 9 | 14 | 14 |
| Day 10 | 14 | 14 |
| Day 11 | 14 | 27 |
| Day 12 | 27 | 27 |
| Day 13 | 27 | 27 |
| Day 14 | 27 | 40 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 7 |
| Day 2 | 0 | 7 |
| Day 3 | 7 | 7 |
| Day 4 | 7 | 7 |
| Day 5 | 7 | 14 |
| Day 6 | 14 | 14 |
| Day 7 | 14 | 14 |
| Day 8 | 14 | 27 |
| Day 9 | 27 | 27 |
| Day 10 | 27 | 27 |
| Day 11 | 27 | 40 |
| Day 12 | 40 | 40 |
| Day 13 | 40 | 40 |
| Day 14 | 40 | 54 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 14 |
| Day 2 | 0 | 14 |
| Day 3 | 14 | 14 |
| Day 4 | 14 | 14 |
| Day 5 | 14 | 27 |
| Day 6 | 27 | 27 |
| Day 7 | 27 | 27 |
| Day 8 | 27 | 40 |
| Day 9 | 40 | 40 |
| Day 10 | 40 | 40 |
| Day 11 | 40 | 54 |
| Day 12 | 54 | 54 |
| Day 13 | 54 | 54 |
| Day 14 | 54 | 81 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9-14 |
| Day 2 | 0 | 9-14 |
| Day 3 | 0 | 9-14 |
| Day 4 | 9-14 | 9-14 |
| Day 5 | 9-14 | 9-14 |
| Day 6 | 9-14 | 18-28 |
| Day 7 | 18-28 | 18-28 |
| Day 8 | 18-28 | 18-28 |
| Day 9 | 18-28 | 18-28 |
| Day 10 | 18-28 | 36-54 |
| Day 11 | 54 | 54 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 10-15 |
| Day 2 | 0 | 10-15 |
| Day 3 | 0 | 10-15 |
| Day 4 | 10-15 | 10-15 |
| Day 5 | 10-15 | 10-15 |
| Day 6 | 10-15 | 20-31 |
| Day 7 | 20-31 | 20-31 |
| Day 8 | 20-31 | 20-31 |
| Day 9 | 20-31 | 20-31 |
| Day 10 | 20-31 | 40-60 |
| Day 11 | 60 | 60 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9 |
| Day 2 | 0 | 9 |
| Day 3 | 0 | 9 |
| Day 4 | 9 | 9 |
| Day 5 | 9 | 9 |
| Day 6 | 9 | 18 |
| Day 7 | 18 | 18 |
| Day 8 | 18 | 18 |
| Day 9 | 18 | 18 |
| Day 10 | 18 | 36 |
| Day 11 | 54 | 54 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 10 |
| Day 2 | 0 | 10 |
| Day 3 | 0 | 10 |
| Day 4 | 10 | 10 |
| Day 5 | 10 | 10 |
| Day 6 | 10 | 20 |
| Day 7 | 20 | 20 |
| Day 8 | 20 | 20 |
| Day 9 | 20 | 20 |
| Day 10 | 20 | 40 |
| Day 11 | 60 | 60 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 14 |
| Day 2 | 0 | 14 |
| Day 3 | 0 | 14 |
| Day 4 | 14 | 14 |
| Day 5 | 14 | 14 |
| Day 6 | 14 | 28 |
| Day 7 | 28 | 28 |
| Day 8 | 28 | 28 |
| Day 9 | 28 | 28 |
| Day 10 | 28 | 54 |
| Day 11 | 54 | 54 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 15 |
| Day 2 | 0 | 15 |
| Day 3 | 0 | 15 |
| Day 4 | 15 | 15 |
| Day 5 | 15 | 15 |
| Day 6 | 15 | 31 |
| Day 7 | 31 | 31 |
| Day 8 | 31 | 31 |
| Day 9 | 31 | 31 |
| Day 10 | 31 | 60 |
| Day 11 | 60 | 60 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9-14 |
| Day 2 | 9-14 | 9-14 |
| Day 3 | 9-14 | 9-14 |
| Day 4 | 9-14 | 18-28 |
| Day 5 | 18-28 | 18-28 |
| Day 6 | 18-28 | 18-28 |
| Day 7 | 18-28 | 36-54 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 10-15 |
| Day 2 | 10-15 | 10-15 |
| Day 3 | 10-15 | 10-15 |
| Day 4 | 10-15 | 20-31 |
| Day 5 | 20-31 | 20-31 |
| Day 6 | 20-31 | 20-31 |
| Day 7 | 20-31 | 40-60 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9 |
| Day 2 | 9 | 9 |
| Day 3 | 9 | 9 |
| Day 4 | 9 | 18 |
| Day 5 | 18 | 18 |
| Day 6 | 18 | 18 |
| Day 7 | 18 | 36 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 10 |
| Day 2 | 10 | 10 |
| Day 3 | 10 | 20 |
| Day 4 | 20 | 20 |
| Day 5 | 20 | 20 |
| Day 6 | 20 | 40 |
| Day 7 | 40 | 60 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 14 |
| Day 2 | 14 | 14 |
| Day 3 | 14 | 28 |
| Day 4 | 28 | 28 |
| Day 5 | 28 | 28 |
| Day 6 | 28 | 54 |
| Day 7 | 54 | 81 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 15 |
| Day 2 | 15 | 15 |
| Day 3 | 15 | 31 |
| Day 4 | 31 | 31 |
| Day 5 | 31 | 31 |
| Day 6 | 31 | 60 |
| Day 7 | 60 | 60 |

In embodiments, elderly patients are administered an Equivalent Amount of Nalbuphine Free Base according to the following titration schedule:

| Day | AM dosage (mg) | PM dosage (mg) |
|---|---|---|
| Day 1 | 0 | 5-8 |
| Day 2 | 0 | 5-8 |
| Day 3 | 0 | 5-8 |
| Day 4 | 5-8 | 5-8 |
| Day 5 | 5-8 | 5-8 |
| Day 6 | 5-8 | 10-15 |
| Day 7 | 10-15 | 10-15 |
| Day 8 | 10-15 | 10-15 |
| Day 9 | 10-15 | 10-15 |
| Day 10 | 10-15 | 20-27 |
| Day 11 | 20-27 | 20-27 |

In embodiments, elderly patients are administered an Equivalent Amount of Nalbuphine Free Base according to the following titration schedule:

| Day | AM dosage (mg) | PM dosage (mg) |
|---|---|---|
| Day 1 | 0 | 2-7 |
| Day 2 | 0 | 2-7 |
| Day 3 | 0 | 2-7 |
| Day 4 | 2-7 | 2-7 |
| Day 5 | 2-7 | 2-7 |
| Day 6 | 2-7 | 9-14 |
| Day 7 | 9-14 | 9-14 |
| Day 8 | 9-14 | 9-14 |
| Day 9 | 9-14 | 9-14 |
| Day 10 | 9-14 | 20-27 |
| Day 11 | 20-27 | 20-27 |

In embodiments, elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 5 |
| Day 2 | 0 | 5 |
| Day 3 | 0 | 5 |
| Day 4 | 10 | 5 |
| Day 5 | 10 | 5 |
| Day 6 | 10 | 10 |
| Day 7 | 10 | 10 |
| Day 8 | 10 | 10 |
| Day 9 | 10 | 10 |
| Day 10 | 10 | 22-27 |
| Day 11 | 22-27 | 22-27 |

In embodiments, elderly patients are administered an Equivalent Amount of Nalbuphine Free Base according to the following titration schedule:

| Day | AM dosage (mg) | PM dosage (mg) |
|---|---|---|
| Day 1 | 0 | 7 |
| Day 2 | 0 | 7 |
| Day 3 | 0 | 7 |
| Day 4 | 7 | 7 |
| Day 5 | 7 | 7 |
| Day 6 | 7 | 14 |
| Day 7 | 14 | 14 |
| Day 8 | 14 | 14 |
| Day 9 | 14 | 14 |
| Day 10 | 14 | 22-27 |
| Day 11 | 22-27 | 22-27 |

In embodiments, elderly patients are administered nalbuphine, or a pharmaceutically acceptable salt thereof according to the following titration schedule:

| Day | AM dosage (mg) | PM dosage (mg) |
|---|---|---|
| Day 1 | 0 | 7 |
| Day 2 | 0 | 7 |
| Day 3 | 0 | 7 |
| Day 4 | 7 | 7 |
| Day 5 | 7 | 7 |
| Day 6 | 7 | 15 |
| Day 7 | 15 | 15 |
| Day 8 | 15 | 15 |
| Day 9 | 15 | 15 |
| Day 10 | 15 | 22-27 |
| Day 11 | 22-27 | 22-27 |

In embodiments, elderly patients are administered an Equivalent Amount of Nalbuphine Free Base according to the following titration schedule:

| Day | AM dosage (mg) | PM dosage (mg) |
|---|---|---|
| Day 1 | 0 | 5-10 |
| Day 2 | 5-10 | 5-10 |
| Day 3 | 5-10 | 5-10 |
| Day 4 | 5-10 | 10-15 |
| Day 5 | 10-15 | 10-15 |
| Day 6 | 10-15 | 10-15 |
| Day 7 | 10-15 | 20-27 |
| Day 8 | 20-27 | 20-27 |

In embodiments, elderly patients are administered nalbuphine, or a pharmaceutically acceptable salt thereof according to the following titration schedule:

| Day | AM dosage (mg) | PM dosage (mg) |
|---|---|---|
| Day 1 | 0 | 4-7 |
| Day 2 | 4-7 | 4-7 |
| Day 3 | 4-7 | 4-7 |
| Day 4 | 4-7 | 9-14 |
| Day 5 | 9-14 | 9-14 |
| Day 6 | 9-14 | 9-14 |
| Day 7 | 9-14 | 18-27 |
| Day 8 | 18-27 | 18-27 |

In embodiments, elderly patients are administered nalbuphine, or a pharmaceutically acceptable salt thereof according to the following titration schedule:

| Day | AM dosage (mg) | PM dosage (mg) |
|---|---|---|
| Day 1 | 0 | 4-7 |
| Day 2 | 0 | 4-7 |
| Day 3 | 4-7 | 4-7 |
| Day 4 | 4-7 | 4-7 |
| Day 5 | 4-7 | 4-7 |
| Day 6 | 4-7 | 4-7 |
| Day 7 | 4-7 | 4-7 |
| Day 8 | 4-7 | 4-7 |
| Day 9 | 4-7 | 4-7 |
| Day 10 | 4-7 | 4-7 |
| Day 11 | 4-7 | 4-7 |
| Day 12 | 4-7 | 4-7 |
| Day 13 | 4-7 | 4-7 |
| Day 14 | 4-7 | 4-7 |

In embodiments, elderly patients are administered nalbuphine, or a pharmaceutically acceptable salt thereof according to the following titration schedule:

| Day | AM dosage (mg) | PM dosage (mg) |
|---|---|---|
| Day 1 | 0 | 4-7 |
| Day 2 | 0 | 4-7 |
| Day 3 | 4-7 | 4-7 |
| Day 4 | 4-7 | 4-7 |
| Day 5 | 4-7 | 4-7 |
| Day 6 | 4-7 | 4-7 |
| Day 7 | 4-7 | 4-7 |
| Day 8 | 4-7 | 4-7 |
| Day 9 | 4-7 | 4-7 |
| Day 10 | 4-7 | 4-7 |
| Day 11 | 4-7 | 4-7 |
| Day 12 | 4-7 | 4-7 |
| Day 13 | 4-7 | 4-7 |
| Day 14 | 4-7 | 9-14 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9-14 |
| Day 2 | 0 | 9-14 |
| Day 3 | 9-14 | 9-14 |
| Day 4 | 9-14 | 9-14 |
| Day 5 | 9-14 | 9-14 |
| Day 6 | 9-14 | 9-14 |
| Day 7 | 9-14 | 9-14 |
| Day 8 | 9-14 | 9-14 |
| Day 9 | 9-14 | 9-14 |
| Day 10 | 9-14 | 9-14 |
| Day 11 | 9-14 | 9-14 |
| Day 12 | 9-14 | 9-14 |
| Day 13 | 9-14 | 9-14 |
| Day 14 | 9-14 | 9-14 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9-14 |
| Day 2 | 0 | 9-14 |
| Day 3 | 9-14 | 9-14 |
| Day 4 | 9-14 | 9-14 |
| Day 5 | 9-14 | 9-14 |
| Day 6 | 9-14 | 9-14 |
| Day 7 | 9-14 | 18-28 |
| Day 8 | 18-28 | 18-28 |
| Day 9 | 18-28 | 18-28 |
| Day 10 | 18-28 | 18-28 |
| Day 11 | 18-28 | 18-28 |
| Day 12 | 18-28 | 18-28 |
| Day 13 | 18-28 | 18-28 |
| Day 14 | 18-28 | 18-28 |

In embodiments, the elderly patients are administered an Equivalent Amount of Nalbuphine Free Base according to the titration schedule provided in the following table:

| Day | AM dosage (mg) | PM dosage (mg) |
|---|---|---|
| Day 1 | 0 | 9-14 |
| Day 2 | 0 | 9-14 |
| Day 3 | 9-14 | 9-14 |
| Day 4 | 9-14 | 9-14 |
| Day 5 | 9-14 | 9-14 |
| Day 6 | 9-14 | 9-14 |
| Day 7 | 9-14 | 18-28 |
| Day 8 | 18-28 | 18-28 |
| Day 9 | 18-28 | 18-28 |
| Day 10 | 18-28 | 18-28 |
| Day 11 | 18-28 | 18-28 |
| Day 12 | 18-28 | 18-28 |
| Day 13 | 18-28 | 18-28 |
| Day 14 | 18-28 | 54 |

In embodiments, the dose of nalbuphine, or a pharmaceutically acceptable salt thereof is not titrated in an elderly patient.

In embodiments, the dosing frequency and dose amount per administration of nalbuphine, or a pharmaceutically acceptable salt thereof, are selected to provide therapeutic effects for the treatment of pruritus. In embodiments, the dosing frequency and dose amount per administration of nalbuphine, or a pharmaceutically acceptable salt thereof are selected to provide therapeutic effects for the treatment of prurigo nodularis. In embodiments, the dosing frequency and dose amount per administration of nalbuphine of a pharmaceutically acceptable salt thereof are selected to provide therapeutic effects for the treatment of uremic pruritus. Methods of treating prurigo nodularis and methods of treating uremic pruritus by administering nalbuphine to a patient in need thereof are described in U.S. Pat. Nos. 8,987,289; 8,637,538; 8,940,753 and 10,238,646 and U.S. Publication Nos. 2018/0125840, 2018/0008592, 2020/0016150 the contents of which are hereby incorporated by reference in their entireties for all purposes.

According to some embodiments of the present disclosure, the dosing frequency and dose amount per administration of nalbuphine, or a pharmaceutically acceptable salt thereof, are selected to provide therapeutic effects for the treatment of cough (such chronic cough), breathlessness, or dyspnea (such as cough, breathlessness or dyspnea associated with IPF). Methods of treating cough (such chronic cough), breathlessness, or dyspnea (such as cough, breathlessness or dyspnea associated with IPF) by administering nalbuphine to a patient in need thereof are described in U.S. Publication No. 2020/0022974, the contents of which are hereby incorporated by reference in their entireties for all purposes.

According to some embodiments of the present disclosure, the dosing frequency and dose amount per administration of nalbuphine, or a pharmaceutically acceptable salt thereof, are selected to provide therapeutic effects for the treatment of dyskinesia. In embodiments, the dosing frequency and dose amount per administration of nalbuphine, or a pharmaceutically acceptable salt thereof, are selected to provide therapeutic effects for the treatment of levodopa-induced dyskinesia (LID). In embodiments, the dosing frequency and dose amount per administration of nalbuphine, or a pharmaceutically acceptable salt thereof, are selected to provide therapeutic effects for the treatment of tardive dyskinesia. In embodiments, the dosing frequency and dose amount per administration of nalbuphine, or a pharmaceutically acceptable salt thereof, are selected to provide therapeutic effects for the treatment of Huntington's disease. Methods of treating dyskinesias (including LID in Parkinson's patients) by administering nalbuphine to a patient in need thereof are described in U.S. Pat. Nos. 9,289,423; 9,918,980; and 10,736,889, the contents of which are hereby incorporated by reference in their entireties for all purposes.

In embodiments, nalbuphine, or a pharmaceutically acceptable salt, solvate or ester thereof, is administered to an elderly patient once a day, twice a day, three times a day, once per week, twice per week, three times per week, five times per week, once every two weeks, one every three weeks, once per month, once every two months, once every three months, or once every six months.

In embodiments, nalbuphine, or a pharmaceutically acceptable salt, solvate or ester thereof, is administered on a once-a-day or twice-a-day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks, including all sub-ranges and values therebetween.

In embodiments, the substantial reduction in itch provided by the methods of the present disclosure requires treatment for a specified time interval (e.g., at least one week) before the patient experiences substantial reduction of itch (i.e., there is an induction period before the patient experiences a substantial reduction in itch). In embodiments, after treatment for at least one week, at least two weeks, at least three weeks, at least four weeks, at least five weeks, at least six weeks, at least seven weeks or at least eight weeks, the patient experiences a substantial reduction of itch compared to prior to the treatment. In embodiments, after treatment for at least one week the patient experiences a substantial reduction of itch compared to prior to the treatment. In embodiment, the substantial reduction in itch may be expressed using any of the methods described herein (for example, decline in worst or average itching intensity Numerical Rating Scale value compared to prior to the treatment, improvement in the ItchyQoL™ scale compared to prior to the treatment, etc.).

In embodiments, the daily dose of nalbuphine, or a pharmaceutically acceptable salt or ester thereof, is in a once or twice daily dose, and then titrated upward until the patient experiences a therapeutic effect. In embodiments, the daily dose can be titrated in increments ranging from about 5 mg to about 60 mg, or about 15 mg to about 60 mg (e.g., about 5 mg, about 10 mg, about 15 mg, about 30 mg or about 60 mg). In embodiments, the daily dose can be titrated in one or more steps. In embodiments, the daily dosage can be titrated by increasing a single daily dosage, or each dose of a twice-daily dosing regimen. In embodiments, the amount a dosage is stepped, where there are multiple titration steps, can be the same, or can be different.

In embodiments, the titration may be initiated with about 7 mg, about 14 mg or about 28 mg of nalbuphine, or a pharmaceutically acceptable salt or ester thereof, once or twice daily. In embodiments, doses can be adjusted in 7 mg or 14 mg increments every 1 to 4 days. In embodiments, the elderly patient patients self-titrate to effect over from about 7 days to about 30 days (for example, from about 12 days to about 20 days) or about 7 days to about 14 days (for example, from about 8 days to about 11 days) to a dose that provides adequate relief from nalbuphine-treatable disorders and minimizes adverse reactions. In embodiments, the titration is conducted for at least about one week, about 2 weeks, about 3 weeks, about 4 weeks or about 5 weeks until a steady state is achieved in the patient.

In embodiments, the titration is initiated with from about 1 mg to about 10 mg of nalbuphine, or a pharmaceutically acceptable salt or ester thereof, once or twice daily including, from about 1 mg, about 2 mg, about 3 mg, about 4 mg, about 5 mg, about 6 mg, about 7 mg, about 8 mg, about 9 mg, or about 10 mg of nalbuphine, or a pharmaceutically acceptable salt or ester thereof, once or twice daily, including all ranges and values therebetween. In embodiments, the dose can be adjusted in from about 2 mg to about 10 mg increments, including form about 2 mg increments, about 3 mg increments, about 4 mg increments, about 5 mg increments, about 6 mg increments, 7 mg increments, about 8 mg increments, about 9 mg increments, or about 10 mg increments, including all ranges and values therebetween. In embodiments, the dose is adjusted every 1-4 days (for example, every day, every 2 days, every 3 days, or every 4 days). In embodiments, the dose is adjusted every 3-4 days. In embodiments, elderly patients can self-titrate to effect over from about 5 days to about 30 days (for example, from about 5 days to about 12 days) to a dose that provides adequate relief from itch and minimizes adverse reactions. In embodiments, the titration is conducted for about or at least about 4 to 14 days (for example about or at least about 4 days, 5 days, 6 days, 7 days, 8 days, 9 days, 10 days, 11 days, 12 days, 13 days, or 14 days). In embodiments, an elderly patient can self-titrate up to from about 9 to about 54 mg of nalbuphine, or a pharmaceutically acceptable salt or ester thereof once or twice daily, including from about 9 mg, about 10 mg, about 15 mg, about 20 mg, about 25 mg, about 30 mg, about 35 mg, about 40 mg, about 45, about 50 mg to about 54 mg including all ranges and values therebetween of nalbuphine, or a pharmaceutically acceptable salt or ester thereof once or twice daily. In embodiments, an elderly patient can be provided with initially about 1-10 mg of nalbuphine, or a pharmaceutically acceptable salt or ester thereof once or twice daily, and titrate up to from about 20-27 mg once or twice daily.

In embodiments, an elderly patient is provided initially with 2 mg, 7 mg or 9 mg tablets to self-titrate to effect up to about 28 mg, about 40 mg, about 54 mg, or about 81 mg, twice a day. In embodiments, the titration dose is started with about 7 mg or about 14 mg, and then gradually increased to about 54 mg or 81 mg twice a day. In embodiments, the titration dose is started with about 14 mg or about 28 mg, and then gradually increased to about 135 mg or 170 mg once a day, e.g., for an elderly patient with a nalbuphine-treatable disorder.

According to some embodiments of the present disclosure, the methods of the present disclosure provide therapeutically effective blood plasma levels of nalbuphine for treating an elderly patient with a nalbuphine-treatable disorder. Blood plasma levels of nalbuphine may be expressed using pharmacokinetic parameters that are known to those skilled in the art, such as steady state plasma levels, AUC, Cₘₐₓ and Cₘᵢₙ. Blood plasma levels of nalbuphine are described in U.S. Publication Nos. 2014/0171459, 2014/0350042, 2015/0359789, and 2017/0216277, which are incorporated by reference herein in their entirety.

In embodiments, the present methods provide steady state plasma levels of nalbuphine that correlate to one or more statistically significant therapeutic effects. In embodiments, the elderly patient has a mean AUCₛₛ (e.g., AUC₀₋₂₄ₕ) of about 80% to about 120% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, or about 120%, including any values or ranges therebetween) of the mean AUCₛₛ (e.g., AUC₀₋₂₄ₕ) of a non-elderly patient treated for the same nalbuphine-treatable disorder following administration of a daily dose of about 40% to about 60% (e.g., about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%, including any values or ranges therebetween) of the daily dose administered to a non-elderly patient.

In embodiments, the elderly patient has a mean Cₘₐₓ of about 80% to about 120% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, or about 120%, including any values or ranges therebetween) of the mean Cₘₐₓ of a non-elderly patient treated for the same nalbuphine-treatable disorder following administration of a daily dose of about 40% to about 60% (e.g., about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%, including any values or ranges therebetween) of the daily dose administered to a non-elderly patient.

In embodiments, the elderly patient has a mean Cₛₛ of about 80% to about 120% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, or about 120%, including any values or ranges therebetween) of the mean Cₛₛ of a non-elderly patient treated for the same nalbuphine-treatable disorder following administration of a daily dose of about 40% to about 60% (e.g., about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%, including any values or ranges therebetween) of the daily dose administered to a non-elderly patient.

In embodiments, the present methods provide steady state plasma levels of nalbuphine that correlate to one or more statistically significant therapeutic effects. In embodiments, the therapeutically effective steady state plasma levels of nalbuphine provided by the methods of the present disclosure is about 10 ng/mL to about 80 ng/ml, including about 20 ng/ml, about 25 ng/ml, about 30 ng/mL, about 35 ng/mL, about 40 ng/ml, about 45 ng/mL, about 50 ng/ml, about 55 ng/ml, about 60 ng/mL, about 65 ng/mL, about 70 ng/ml, about 75 ng/ml or about 80 ng/mL, including all ranges there between.

In embodiments, the present methods provide mean steady state AUC₀₋₂₄ₕ (expressed in terms of ng*hr/mL) levels of nalbuphine that correlate to one or more statistically significant therapeutic effects. In embodiments, the therapeutically effective mean steady state AUC₀₋₂₄ₕ levels of nalbuphine provided by the methods of the present disclosure are about 90 ng*hr/mL to about 1600 ng*hr/mL, including about 100 ng*hr/mL, about 200 ng*hr/mL, about 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, and about 1500 ng*hr/mL, including all ranges there between.

In embodiments, the present methods provide a mean AUC₀₋₂₄ₕ from about 40 ng·hr/mL to about 1600 ng·hr/mL. In embodiments, the present methods provide a mean AUC₀₋₂₄ₕ from about 80 ng·hr/mL to about 1600 ng·hr/mL. In embodiments, the present methods provide a mean AUC₀₋₂₄ₕ from about 80 ng·hr/mL to about 400 ng·hr/mL. In embodiments, the present methods provide a mean AUC₀₋₂₄ₕ from about 360 ng·hr/mL to about 620 ng·hr/mL. In embodiments, the present methods provide a mean AUC₀₋₂₄ₕ from about 200 ng·hr/mL to about 800 ng·hr/mL. In embodiments, the present methods provide a mean AUC₀₋₂₄ₕ from about 400 ng·hr/mL to about 1000 ng·hr/mL. In embodiments, the present methods provide a mean AUC₀₋₂₄ₕ from about 600 ng·hr/mL to about 1200 ng·hr/mL. In embodiments, the present methods provide a mean AUC₀₋₂₄ₕ from about 800 ng·hr/mL to about 1400 ng·hr/mL. In embodiments, the present methods provide a mean AUC₀₋₂₄ₕ from about 1000 ng·hr/mL to about 1600 ng·hr/mL. In embodiments, the present methods provide a mean AUC₀₋₂₄ₕ from about 200 ng·hr/mL to about 600 ng·hr/mL. In embodiments, the present methods provide a mean AUC₀₋₂₄ₕ from about 400 ng·hr/mL to about 800 ng·hr/mL. In embodiments, the present methods provide a mean AUC₀₋₂₄ₕ from about 600 ng·hr/mL to about 1000 ng·hr/mL. In embodiments, the present methods provide a mean AUC₀₋₂₄ₕ from about 800 ng·hr/mL to about 1200 ng·hr/mL. In embodiments, the present methods provide a mean AUC₀₋₂₄ₕ from about 1000 ng·hr/mL to about 1400 ng·hr/mL. In embodiments, the present methods provide a mean AUC₀₋₂₄ₕ from about 1200 ng·hr/mL to about 1600 ng·hr/mL. In embodiments, the present methods provide a mean AUC₀₋₂₄ₕ from about 85 ng·hr/mL to about 1600 ng·hr/mL. In embodiments, the present methods provide a mean AUC₀₋₂₄ₕ from about 440 ng·hr/mL to about 1245 ng·hr/mL. In embodiments, the present methods provide a mean AUC₀₋₂₄ₕ from about 80 h*ng/mL to about 1600 h*ng/mL.

In embodiments, the present methods provide a mean AUCₜₐᵤ from about 20 ng·hr/mL to about 800 ng·hr/mL. In embodiments, the present methods provide a mean AUCₜₐᵤ from about 40 ng·hr/mL to about 800 ng·hr/mL. In embodiments, the present methods provide a mean AUCₜₐᵤ from about 40 ng·hr/mL to about 200 ng·hr/mL. In embodiments, the present methods provide a mean AUCₜₐᵤ from about 180 ng·hr/mL to about 320 ng·hr/mL In embodiments, the present methods provide a mean AUCₜₐᵤ from about 180 ng·hr/mL to about 320 ng·hr/mL. In embodiments, the present methods provide a mean AUCₜₐᵤ from about 100 ng·hr/mL to about 400 ng·hr/mL. In embodiments, the present methods provide a mean AUCₜₐᵤ from about 200 ng·hr/mL to about 500 ng·hr/mL. In embodiments, the present methods provide a mean AUCₜₐᵤ from about 300 ng·hr/mL to about 600 ng·hr/mL. In embodiments, the present methods provide a mean AUCₜₐᵤ from about 400 ng·hr/mL to about 700 ng·hr/mL. In embodiments, the present methods provide a mean AUCₜₐᵤ from about 500 ng·hr/mL to about 800 ng·hr/mL. In embodiments, the present methods provide a mean AUCₜₐᵤ from about 100 ng·hr/mL to about 300 ng·hr/mL. In embodiments, the present methods provide a mean AUCₜₐᵤ from about 200 ng·hr/mL to about 400 ng·hr/mL. In embodiments, the present methods provide a mean AUCₜₐᵤ from about 300 ng·hr/mL to about 500 ng·hr/mL. In embodiments, the present methods provide a mean AUCₜₐᵤ from about 400 ng·hr/mL to about 600 ng·hr/mL. In embodiments, the present methods provide a mean AUCₜₐᵤ from about 500 ng·hr/mL to about 700 ng·hr/mL. In embodiments, the present methods provide a mean AUCₜₐᵤ from about 600 ng·hr/mL to about 800 ng·hr/mL. In embodiments, the present methods provide a mean AUCₜₐᵤ from about 43.2 h*ng/ml to about 769.99 h*ng/ml. In embodiments, the present methods provide a mean AUCₜₐᵤ from about 221.68 h*ng/ml to about 621.79 h*ng/ml. In embodiments, the present methods provide a mean AUCₜₐᵤ from about 40 h*ng/ml to about 800 h*ng/mL.

In embodiments, the present disclosure provides kits for use in treating a nalbuphine treatable disorder (e.g., as described herein). Such kits comprise nalbuphine or a pharmaceutical composition comprising nalbuphine and a pharmaceutically acceptable carrier (e.g., as described herein). The kits of the present disclosure may be used for administering nalbuphine at different dosage intervals, or for titrating nalbuphine according to methods described herein. In embodiments, the kits of the present disclosure may comprise directions for administration. For example, the kit can include instructions to administer nalbuphine in a suitable manner to perform the methods described herein, e.g., in a suitable dose, dosage form, dosing intervals (e.g., as described herein). In embodiments, the informational material can include instructions to administer the nalbuphine to a suitable subject, e.g., an elderly patient as described herein.

The kit can include one or more containers for the compositions as described herein. In embodiments, the kit contains separate containers, dividers or compartments for the composition and informational material. For example, the composition can be contained in a bottle, vial, or syringe. In embodiments, the separate elements of the kit are contained within a single, undivided container. For example, the composition is contained in a bottle, vial or syringe that has attached thereto the informational material in the form of a label. In embodiments, the kit comprises a plurality (e.g., a pack) of individual containers, each containing one or more unit dosage forms (e.g., a dosage form described herein) of a composition described herein. For example, the kit can include a plurality of syringes, ampules, or foil packets each containing a single unit dose of a composition described herein. An example of such a kit is a blister pack, as typically used for the packaging of tablets, capsules and the like. The containers of the kits can be air tight, waterproof (e.g., impermeable to changes in moisture or evaporation), and/or light-tight.

In embodiments, provided herein is a kit for treating a nalbuphine-treatable disorder (e.g., as described herein) in an elderly patient, comprising one or more AM and PM dosages of about 7 mg to about 85 mg of an Equivalent Amount of Nalbuphine Free Base. In embodiments, provided herein is a kit for treating a nalbuphine-treatable disorder (e.g., as described herein) in an elderly patient, comprising one or more AM and PM dosages of nalbuphine or a pharmaceutically acceptable salt thereof selected from the group consisting of about 3 mg, about 4 mg, about 5 mg, about 7 mg, about 8 mg, about 9 mg, about 10 mg, about 14 mg, about 15 mg, about 20 mg, about 27 mg, about 31 mg, about 40 mg, about 54 mg, about 60 mg, and about 85 mg of an Equivalent Amount of Nalbuphine Free Base, including all sub-ranges and values therebetween.

In embodiments of the kits disclosed herein, the AM and PM dosage units are to be administered for about, or at least about 7 to about 30 days. In embodiments of the kits disclosed herein, the AM and PM dosage units are to be administered for about, or at least about 7 to about 14 days. In embodiments, the AM and PM dosage units are to be administered for about, or at least about 17 days. In embodiments, the AM and PM dosage units are to be administered for about, or at least about 14 days. In embodiments, the AM and PM dosage units are to be administered for about 11 days. In embodiments, the AM and PM dosage units are to be administered for about, or at least about 8 days. In embodiments, the AM dosage unit is the same as the PM dosage unit. In embodiments, the AM dosage unit is the different to the PM dosage unit.

In embodiments, the present disclosure provides a kit for treating a nalbuphine-treatable disorder (e.g., as described herein) in an elderly patient, the kit comprising AM and PM dosage units of nalbuphine to be administered for 14 days to the patient, wherein the AM and PM dosage units are as shown in the following tables:

| Day | AM dosage (mg) | PM dosage (mg) |
|---|---|---|
| Day 1 | 0 | 3 |
| Day 2 | 0 | 3 |
| Day 3 | 3 | 3 |
| Day 4 | 3 | 3 |
| Day 5 | 3 | 7 |
| Day 6 | 7 | 7 |
| Day 7 | 7 | 7 |
| Day 8 | 7 | 14 |
| Day 9 | 14 | 14 |
| Day 10 | 14 | 14 |
| Day 11 | 14 | 27 |
| Day 12 | 27 | 27 |
| Day 13 | 27 | 27 |
| Day 14 | 27 | 40 |

| Day | AM dosage (mg) | PM dosage (mg) |
|---|---|---|
| Day 1 | 0 | 7 |
| Day 2 | 0 | 7 |
| Day 3 | 7 | 7 |
| Day 4 | 7 | 7 |
| Day 5 | 7 | 14 |
| Day 6 | 14 | 14 |
| Day 7 | 14 | 14 |
| Day 8 | 14 | 27 |
| Day 9 | 27 | 27 |
| Day 10 | 27 | 27 |
| Day 11 | 27 | 40 |
| Day 12 | 40 | 40 |
| Day 13 | 40 | 40 |
| Day 14 | 40 | 54 |

In embodiments, the present disclosure provides a kit for treating a nalbuphine-treatable disorder (e.g., as described herein) in an elderly patient, the kit comprising AM and PM dosage units of nalbuphine to be administered for 17 days to the patient, wherein the AM and PM dosage units are as shown in the following tables:

| Day | AM dosage (mg) | PM dosage (mg) |
|---|---|---|
| Day 1 | 0 | 14 |
| Day 2 | 0 | 14 |
| Day 3 | 14 | 14 |
| Day 4 | 14 | 14 |
| Day 5 | 14 | 27 |
| Day 6 | 27 | 27 |
| Day 7 | 27 | 27 |
| Day 8 | 27 | 40 |
| Day 9 | 40 | 40 |
| Day 10 | 40 | 40 |
| Day 11 | 40 | 54 |
| Day 12 | 54 | 54 |
| Day 13 | 54 | 54 |
| Day 14 | 54 | 85 |
| Day 15 | 85 | 85 |
| Day 16 | 85 | 85 |
| Day 17 | 85 | 85 |

In embodiments, the present disclosure provides a kit for treating a nalbuphine-treatable disorder (e.g., as described herein) in an elderly patient, the kit comprising AM and PM dosage units of nalbuphine to be administered for 11 days to the patient, wherein the AM and PM dosage units are as shown in the following tables:

| Day | AM dosage (mg) | PM dosage (mg) |
|---|---|---|
| Day 1 | 0 | 10-15 |
| Day 2 | 0 | 10-15 |
| Day 3 | 0 | 10-15 |
| Day 4 | 10-15 | 10-15 |
| Day 5 | 10-15 | 10-15 |
| Day 6 | 10-15 | 20-31 |
| Day 7 | 20-31 | 20-31 |
| Day 8 | 20-31 | 20-31 |
| Day 9 | 20-31 | 20-31 |
| Day 10 | 20-31 | 40-60 |
| Day 11 | 60 | 60 |

| Day | AM dosage (mg) | PM dosage (mg) |
|---|---|---|
| Day 1 | 0 | 10 |
| Day 2 | 0 | 10 |
| Day 3 | 0 | 10 |
| Day 4 | 10 | 10 |
| Day 5 | 10 | 10 |
| Day 6 | 10 | 20 |
| Day 7 | 20 | 20 |
| Day 8 | 20 | 20 |
| Day 9 | 20 | 20 |
| Day 10 | 20 | 40 |
| Day 11 | 60 | 60 |

| Day | AM dosage (mg) | PM dosage (mg) |
|---|---|---|
| Day 1 | 0 | 15 |
| Day 2 | 0 | 15 |
| Day 3 | 0 | 15 |
| Day 4 | 15 | 15 |
| Day 5 | 15 | 15 |
| Day 6 | 15 | 31 |
| Day 7 | 31 | 31 |
| Day 8 | 31 | 31 |
| Day 9 | 31 | 31 |
| Day 10 | 31 | 60 |
| Day 11 | 60 | 60 |

In embodiments, the present disclosure provides a kit for treating a nalbuphine-treatable disorder (e.g., as described herein) in an elderly patient, the kit comprising AM and PM dosage units of nalbuphine to be administered for 8 days to the patient, wherein the AM and PM dosage units are as shown in the following tables:

| Day | AM dosage (mg) | PM dosage (mg) |
|---|---|---|
| Day 1 | 0 | 10-15 |
| Day 2 | 10-15 | 10-15 |
| Day 3 | 10-15 | 10-15 |
| Day 4 | 10-15 | 20-31 |
| Day 5 | 20-31 | 20-31 |
| Day 6 | 20-31 | 20-31 |
| Day 7 | 20-31 | 40-60 |
| Day 8 | 60 | 60 |

| Day | AM dosage (mg) | PM dosage (mg) |
|---|---|---|
| Day 1 | 0 | 10 |
| Day 2 | 10 | 10 |
| Day 3 | 10 | 10 |
| Day 4 | 10 | 20 |
| Day 5 | 20 | 20 |
| Day 6 | 20 | 20 |
| Day 7 | 20 | 40 |
| Day 8 | 60 | 60 |

| Day | AM dosage (mg) | PM dosage (mg) |
|---|---|---|
| Day 1 | 0 | 15 |
| Day 2 | 15 | 15 |
| Day 3 | 15 | 15 |
| Day 4 | 15 | 31 |
| Day 5 | 31 | 31 |
| Day 6 | 31 | 31 |
| Day 7 | 31 | 60 |
| Day 8 | 60 | 60 |

In embodiments, the present disclosure provides a kit for treating a nalbuphine-treatable disorder (e.g., as described herein) in an elderly patient, the kit comprising AM and PM dosage units of nalbuphine to be administered for 11 days to the patient, wherein the AM and PM dosage units are as shown in the following tables:

| Day | AM dosage (mg) | PM dosage (mg) |
|---|---|---|
| Day 1 | 0 | 5-8 |
| Day 2 | 0 | 5-8 |
| Day 3 | 0 | 5-8 |
| Day 4 | 5-8 | 5-8 |
| Day 5 | 5-8 | 5-8 |
| Day 6 | 5-8 | 10-15 |
| Day 7 | 10-15 | 10-15 |
| Day 8 | 10-15 | 10-15 |
| Day 9 | 10-15 | 10-15 |
| Day 10 | 10-15 | 20-27 |
| Day 11 | 20-27 | 20-27 |

| Day | AM dosage (mg) | PM dosage (mg) |
|---|---|---|
| Day 1 | 0 | 5 |
| Day 2 | 0 | 5 |
| Day 3 | 0 | 5 |
| Day 4 | 10 | 5 |
| Day 5 | 10 | 5 |
| Day 6 | 10 | 10 |
| Day 7 | 10 | 10 |
| Day 8 | 10 | 10 |
| Day 9 | 10 | 10 |
| Day 10 | 10 | 22-27 |
| Day 11 | 22-27 | 22-27 |

| Day | AM dosage (mg) | PM dosage (mg) |
|---|---|---|
| Day 1 | 0 | 7 |
| Day 2 | 0 | 7 |
| Day 3 | 0 | 7 |
| Day 4 | 7 | 7 |
| Day 5 | 7 | 7 |
| Day 6 | 7 | 15 |
| Day 7 | 15 | 15 |
| Day 8 | 15 | 15 |
| Day 9 | 15 | 15 |
| Day 10 | 15 | 22-27 |
| Day 11 | 22-27 | 22-27 |

In embodiments, the present disclosure provides a kit for treating a nalbuphine-treatable disorder (e.g., as described herein) in an elderly patient, the kit comprising: AM and PM dosage units of nalbuphine to be administered for 8 days to the patient, wherein the AM and PM dosage units are as shown in the following table:

| Day | AM dosage (mg) | PM dosage (mg) |
|---|---|---|
| Day 1 | 0 | 4-7 |
| Day 2 | 4-7 | 4-7 |
| Day 3 | 4-7 | 4-7 |
| Day 4 | 4-7 | 9-14 |
| Day 5 | 9-14 | 9-14 |
| Day 6 | 9-14 | 9-14 |
| Day 7 | 9-14 | 18-27 |
| Day 8 | 18-27 | 18-27 |

According to some embodiments of the present disclosure, administering of nalbuphine, or a pharmaceutically acceptable salt, solvate or ester thereof, provides statistically significant therapeutic effect. In embodiments, the statistically significant therapeutic effect is determined based on one or more standards or criteria provided by one or more regulatory agencies in the United States, e.g., FDA or other countries. In embodiments, the statistically significant therapeutic effect is determined based on results obtained from regulatory agency approved clinical trial set up and/or procedure.

In embodiments, the statistically significant therapeutic effect is determined based on a patient population of at least 20, 50, 60, 100, 200, 300, 400, 500, 600, 700, 800, 900, 1000 or 2000. In embodiments, the statistically significant therapeutic effect is determined based on data obtained from randomized and double blinded clinical trial set up. In embodiments, the statistically significant therapeutic effect is determined based on data with a p value of less than or equal to about 0.05, 0.04, 0.03, 0.02 or 0.01. In embodiments, the statistically significant therapeutic effect is determined based on data with a confidence interval greater than or equal to 95%, 96%, 97%, 98% or 99%. In embodiments, the statistically significant therapeutic effect is determined on approval of Phase III clinical trial of the methods provided by the present disclosure, e.g., by FDA in the US.

In embodiments, the methods of the present disclosure provide treating a nalbuphine-treatable disorder in an elderly patient by administering an effective dose of nalbuphine, wherein the methods reduce the severity of the symptoms of the nalbuphine-treatable disorder during or after the administration period for at least 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the severity of the symptoms prior to administration.

In embodiments, the statistically significant therapeutic effect is determined by a randomized double blind clinical trial of patients treated with nalbuphine, or a pharmaceutically acceptable salt, solvate or ester thereof, and optionally in combination with standard care. In embodiments, the statistically significant therapeutic effect is determined by a randomized clinical trial and using Numerical Rating Scale (NRS) as primary efficacy parameter and optionally in combination with any other commonly accepted criteria for pruritus assessment.

In embodiments, the NRS score of the patient during the administration period is less than the NRS score of the patient prior to treatment. In embodiments, the NRS score of the patient during or after the administration period is at least about 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the NRS score of the patient prior to treatment. In embodiments, the NRS score of the patient during the administration period is at least about 1 point less (e.g., at least about 1, at least about 1.5, at least about 2, at least about 2.5, at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, or about 10, including any values or ranges therebetween) than the NRS score of the patient prior to treatment. In embodiments, the average NRS score of the patient during the administration period is at least about 0.1 point less (e.g., at least about 0.1, at least about 0.2, at least about 0.3, at least about 0.4, at least about 0.5, at least about 0.6, at least about 0.7, at least about 0.8, at least about 0.9, at least about 1, including any values or ranges therebetween) than the NRS score of the patient prior to treatment.

In embodiments, the average NRS score of the elderly patient during the administration period is about the same or at least about 0.1 point lower (e.g., at least about 0.1, at least about 0.2, at least about 0.3, at least about 0.4, at least about 0.5, at least about 0.6, at least about 0.7, at least about 0.8, at least about 0.9, at least about 1, including any values or ranges therebetween) than the average NRS score of an otherwise physiologically identical non-elderly patient. In embodiments, the average NRS score of the elderly patient during the administration period is about the same or at least about 1 point less (e.g., at least about 1, at least about 1.5, at least about 2, at least about 2.5, at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, or about 10, including any values or ranges therebetween) than the average NRS score of an otherwise physiologically identical non-elderly patient. In embodiments, the average NRS score of the elderly patient during the administration period is about the same as the average NRS score of an otherwise physiologically identical non-elderly patient. In embodiments, the average NRS score of the elderly patient during the administration period is at least about 0.5 point lower than the average NRS score of an otherwise physiologically identical non-elderly patient.

In general, statistical analysis can include any suitable method permitted by a regulatory agency, e.g., FDA in the US or Europe or any other country. In embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

The embodiments described herein should be understood to be illustrative of the present disclosure and should not be construed as limiting. On the contrary, the present disclosure embraces alternatives and equivalents thereof, as embodied by the appended claims. Each reference disclosed herein is incorporated by reference herein in its entirety.

The following non-limiting example illustrates various aspects of the present disclosure.

### EXAMPLE

### Example 1: Evaluation of PK Parameters in Elderly Patients Chronically Administered Nalbuphine

Nalbuphine pharmacokinetics were computationally modelled using samples collected during two clinical trials: NCT02373215 and NCT02174419. Age was included as a covariate in the PK model.

Total systemic clearance rate of nalbuphine decreases with age following oral administration of nalbuphine resulting in higher absolute bioavailability (˜44%) in subjects aged older than 65 years compared to non-elderly subjects (11%). The PK modelling data showed an approximately 5-fold increase in the mean steady state AUC (AUC_{0-24,ss}) in patients aged 72 years old or more compared to non-elderly patients (i.e., 23 years old or less). These data suggest that elderly patients will require dose adjustment of nalbuphine compared to non-elderly patients treated with nalbuphine for the same condition. The following table summarizes the data analysis.

Observation/Modeling

Slower clearance of nalbuphine (e.g., CL/F reduction) with increase in age
Relative to median age of 56, estimates:
28% increase in AUC by age 75
33% decrease in AUC at age 35

### Example 2: PK-PD Modeling and Analysis in Elderly Patients after Nalbuphine Treatment

The nalbuphine PK-PD model of Example 1 was further analyzed. The PK of nalbuphine was characterized by a one compartment model with first order absorption and elimination. Apparent clearance of plasma nalbuphine (95% CI) was calculated at 236 L/h (208 to 264 L/h). There was no observable effect on CL/F due to race (Caucasian vs African American) or BMI. Clearance was affected only by the age of the patients.

As noted above, age has been identified as a significant variable in nalbuphine exposure. The age-effect was significant in adult patients with uremic pruritus on hemodialysis, but similar observations have also been reported in healthy subjects. Following intravenous (IV) administration, elimination half-life in children between 1.5 and 8.5 yrs. was significantly faster (T½=0.9 h) than in young adults 23-32 yr of age (T½=1.96 h) or elderly subjects (65-90 yrs., T½=2.3 hr). The volume of distribution was comparable across the age groups.

The effect of age on exposure and simulated NRS in the HD population is presented in Table 1. The estimated effect of age on clearance and exposure (AUCₛₛ) indicated that clearance in younger adults is 2-fold faster than in older patients resulting in nearly double the exposure. The increase in nalbuphine exposure in the older patients resulted in a greater decrease in the estimated worst itch Numeric Rating Scale (NRS) (Table 1, estimated NRS change from the average baseline NRS of 6.8).

TABLE 1
Effect of Age on Clearance and Exposure
and NRS Based on PK-PD Model

| | | Half- | | Estimated NRS |
|---|---|---|---|---|
| | Clearance | life | AUCss | change |
| Age (yr) | (L/hr) | (hr) | (ng*hr/ml) | NRS BSL = 6.8 |
| 35 | 338.95 | 9.22 | 354.03 | −3.85 |
| 45 | 273.55 | 11.43 | 438.67 | −3.98 |
| 56\hat{ } | 227.00 | 13.77 | 520.57 | −4.09 |
| 65 | 199.90 | 15.63 | 528.63 | −4.19 |
| 75 | 176.93 | 17.66 | 600.30 | −4.27 |
| 85 | 159.01 | 19.8 | 754.65 | −4.33 |

\hat{ }median age in study, range 23-89 years. Median weight: 83.3 kg; range: 39.4-180.7 kg.
BSL: baseline NRS score.
AUCss: area under the plasma concentration-time curve at steady state

What is claimed is:

1. A method of treating a nalbuphine-treatable disorder in a patient in need thereof, comprising:
administering a therapeutically effective daily dose of nalbuphine to an elderly patient, wherein the administered daily dose is about 40% to about 60% of the daily dose administered to a non-elderly patient treated for the same nalbuphine-treatable disorder.

2. The method of claim 1, wherein the administered daily dose is about 50% of the daily dose administered to a non-elderly patient treated for the same nalbuphine-treatable disorder.

3. The method of claim 1 or 2, wherein the nalbuphine-treatable disorder is idiopathic pulmonary fibrosis (IPF) cough, uremic pruritus, prurigo nodularis, and levodopa-induced dyskinesia (LID).

4. The method of claim 3, wherein the nalbuphine-treatable disorder is prurigo nodularis.

5. The method of claim 3, wherein the nalbuphine-treatable disorder is uremic pruritus.

6. The method of any one of claims 1-5, wherein the non-elderly patient is administered a daily dose of about 54 mg to about 324 mg of an Equivalent Amount of Nalbuphine Free Base.

7. The method of any one of claims 1-6, wherein the elderly patient is administered a daily dose of about 27 mg to about 170 mg of an Equivalent Amount of Nalbuphine Free Base.

8. The method of claim 3, wherein the nalbuphine-treatable disorder is idiopathic pulmonary fibrosis (IPF) cough.

9. The method of claim 8, wherein the daily dose administered to a non-elderly patient is about 54 mg to about 216 mg of an Equivalent Amount of Nalbuphine Free Base.

10. The method of claim 8, wherein the daily dose administered to a non-elderly patient is about 54 mg to about 324 mg of an Equivalent Amount of Nalbuphine Free Base.

11. The method of any one of claims 8-10, wherein the daily dose administered to an elderly patient is about 27 mg to about 216 mg of an Equivalent Amount of Nalbuphine Free Base.

12. The method of any one of claims 1-11, wherein the nalbuphine is administered twice daily.

13. The method of any one of claims 1-11, wherein the nalbuphine is administered once daily.

14. The method of any one of claims 1-13, further comprising titrating the dose for about 7 to 30 days.

15. The method of any one of claims 1-14, further comprising titrating the dose for about 7 to 14 days.

16. The method of claim 15, wherein the titration comprises administration of an Equivalent Amount of Nalbuphine Free Base according to the following schedule

| Day | AM dosage (mg) | PM dosage (mg |
|---|---|---|
| Day 1 | 0 | about 14 |
| Day 2 | 0 | about 14 |
| Day 3 | about 14 | about 14 |
| Day 4 | about 14 | about 14 |
| Day 5 | about 14 | about 27 |
| Day 6 | about 27 | about 27 |
| Day 7 | about 27 | about 27 |
| Day 8 | about 27 | about 40 |
| Day 9 | about 40 | about 40 |
| Day 10 | about 40 | about 40 |
| Day 11 | about 40 | about 54 |
| Day 12 | about 54 | about 54 |
| Day 13 | about 54 | about 54 |
| Day 14 | about 54 | about 81. |

17. The method of any one of claims 1-16, wherein the elderly patient is at least 75 years old.

18. The method of any one of claims 1-17, wherein the non-elderly patient is 18 to 74 years old.

19. The method of any one of claims 1-16, wherein the elderly patient is at least 65 years old.

20. The method of any one of claims 1-16 and 19, wherein the non-elderly patient is 18 to 64 years old.

21. A method of treating a nalbuphine-treatable disorder in a patient in need thereof, comprising:
(a) determining the patient's age;
(b) administering a daily dose of about 28 mg to about 170 mg of an Equivalent Amount of Nalbuphine Free Base to an elderly patient; and
(c) administering a daily dose of about 54 mg to about 324 mg of an Equivalent Amount of Nalbuphine Free Base to a non-elderly patient, wherein nalbuphine treatable conditions is idiopathic pulmonary fibrosis (IPF) cough, uremic pruritus, prurigo nodularis, or levodopa-induced dyskinesia (LID).

22. The method of claim 21, wherein the daily dose administered to an elderly patient is about 40% to about 60% of the daily dose administered to a non-elderly patient treated for the same nalbuphine-treatable disorder.

23. The method of claim 21 or 22, wherein the daily dose administered to an elderly patient is about 50% of the daily dose administered to a non-elderly patient treated for the same nalbuphine-treatable disorder.

24. The method of any one of claims 21-23, wherein the nalbuphine-treatable disorder is prurigo nodularis.

25. The method of any one of claims 21-23, wherein the nalbuphine-treatable disorder is uremic pruritus.

26. The method of any one of claims 21-25, wherein the elderly patient is administered a daily dose of about 28 mg to about 170 mg of an Equivalent Amount of Nalbuphine Free Base.

27. The method of any one of claims 21-23, wherein the nalbuphine-treatable disorder is idiopathic pulmonary fibrosis (IPF) cough.

28. The method of claim 27, wherein the daily dose administered to an elderly patient is about 27 mg to about 108 mg of an Equivalent Amount of Nalbuphine Free Base.

29. The method of claim 27, wherein the daily dose administered to an elderly patient is about 27 mg to about 216 mg of an Equivalent Amount of Nalbuphine Free Base.

30. The method of any one of claims 21-29, wherein the nalbuphine is administered twice daily.

31. The method of any one of claims 21-30, wherein the nalbuphine is administered once daily.

32. The method of any one of claims 21-31, further comprising titrating the dose for about 7 to 30 days.

33. The method of any one of claims 21-32, further comprising titrating the dose for about 7 to 14 days.

34. The method of claim 33, wherein the titration comprises administration of an Equivalent Amount of Nalbuphine Free Base according to the following schedule

| Day | AM dosage (mg) | PM dosage (mg) |
|---|---|---|
| Day 1 | 0 | about 14 |
| Day 2 | 0 | about 14 |
| Day 3 | about 14 | about 14 |
| Day 4 | about 14 | about 14 |
| Day 5 | about 14 | about 27 |
| Day 6 | about 27 | about 27 |
| Day 7 | about 27 | about 27 |
| Day 8 | about 27 | about 40 |
| Day 9 | about 40 | about 40 |
| Day 10 | about 40 | about 40 |
| Day 11 | about 40 | about 54 |
| Day 12 | about 54 | about 54 |
| Day 13 | about 54 | about 54 |
| Day 14 | about 54 | about 81. |

35. The method of any one of claims 21-34, wherein the elderly patient is at least 75 years old.

36. The method of any one of claims 21-35, wherein the non-elderly patient is 18 to 74 years old.

37. The method of any one of claims 21-34, wherein the elderly patient is at least 65 years old.

38. The method of any one of claims 21-34 and 37, wherein the non-elderly patient is 18 to 64 years old.
